(19) United States
(12) Patent Application Publication
(10) Pub. No.: US 2024/0087685 A1
(43) Pub. Date: Mar. 14, 2024

(54) SYSTEMS AND METHODS FOR EVALUATION OF STRUCTURE AND PROPERTY OF POLYNUCLEOTIDES

(71) Applicant: THE TEXAS A&M UNIVERSITY SYSTEM, College Station, TX (US)

(72) Inventors: Qing Sun, College Station, TX (US); Shujun He, College Station, TX (US)

(21) Appl. No.: 18/273,293

(22) PCT Filed: Jan. 20, 2022
(86) PCT No.: PCT/US22/13203

§ 371 Date: Jul. 20, 2023

Related U.S. Application Data
(60) Provisional application No. 63/199,721, filed on Jan. 20, 2021.

(51) Int. Cl. G16B 40/20; G06N 3/0455; G16B 40/30

(52) U.S. Cl. CPC G16B 40/20; G06N 3/0455; G16B 40/30

(57) ABSTRACT

Provided here are neural network-based methods and systems that process nucleic acid sequences to identify, characterize, and interpret specific properties, like characterization of promoters of gene sequences, identification of viral genomes, and stability of the nucleic acids. The neural network models can be trained in supervised, unsupervised, and semi-supervised environments.

### CROSS-REFERENCE TO RELATED APPLICATIONS

This application claims the benefit of and priority to U.S. Provisional Application No. 63/199,721, filed on Jan. 20, 2021, which is incorporated by reference herein in its entirety.

### TECHNICAL FIELD

This disclosure relates to methods and systems for evaluating structure and property of polynucleotides, including deoxyribonucleic acid (DNA) and ribonucleic acid (RNA). These properties can include stability, function as a promoter or enhancer, and virality or infectiousness.

### BACKGROUND

Nucleic acids, especially DNA and RNA, are essential parts of any biological system. Information stored in DNA and RNA provides instructions on how the complex functions should be carried out in a biological organism. However, understanding the instructions stored in DNA and RNA has always been challenging. Even though the sequences that encode amino acids, which eventually form proteins, have been mapped, the functions of many non-coding DNA and RNA sequences still remain largely unknown. Experimental approach is often used to understand the specific function of a DNA or RNA sequence through genetic mutation, but this approach is both time consuming and labor intensive due to the tremendous amount of manipulations and data required to provide meaningful information.

### SUMMARY

Applicant has recognized that the need for machine assisted systems and methods for evaluation of structure and property of nucleic acids. Provided here are computer-implemented methods for generating and using a neural network model for determining a property of a polynucleotide sequence. One embodiment includes a computer-implemented method for determining a property of a polynucleotide sequence that includes the steps of: receiving a plurality of nucleotides organized by their position defining a polynucleotide sequence and a request for determining a property of the polynucleotide sequence; and applying a deep learning prediction model to the plurality of nucleotides to determine the property of at least a portion of the polynucleotide sequence. The deep learning prediction model applies a trained convolutional neural network and a self-attention mechanism to capture local and global dependencies of each of the plurality of nucleotides and the polynucleotide sequence. The convolutional neural network can be trained in one or more of a supervised, unsupervised, and semi-supervised learning environments. The property of the portion of the polynucleotide sequence can be the presence of a promoter or an enhancer sequence for gene expression. The property of the portion of the polynucleotide sequence can be the presence of a viral gene. The property of the portion of the polynucleotide sequence can be the stability of the polynucleotide sequence under an environmental parameter. The environmental parameter can be one or more of temperature, pH, or presence of a nuclease. Embodiments also include one or more non-transitory, computer-readable medium having computer executable instructions that implement the foregoing computer-implemented methods.

Embodiments also include systems with one or more processors coupled to a memory, which is loaded with computer instructions to perform a computer-implemented method for determining a property of a polynucleotide sequence. These computer instructions, when executed on the one or more processors implement actions that include: receiving a plurality of nucleotides organized by their position defining a polynucleotide sequence and a request for determining a property of the polynucleotide sequence; and applying a deep learning prediction model to the plurality of nucleotides to determine the property of at least a portion of the polynucleotide sequence. The deep learning prediction model applies a trained convolutional neural network and a self-attention mechanism to capture local and global dependencies of each of the plurality of nucleotides and the polynucleotide sequence. The convolutional neural network can be trained in one or more of a supervised, unsupervised, and semi-supervised learning environments. The property of the portion of the polynucleotide sequence can be the presence of a promoter or an enhancer sequence for gene expression. The property of the portion of the polynucleotide sequence can be the presence of a viral gene. The property of the portion of the polynucleotide sequence can be the stability of the polynucleotide sequence under an environmental parameter. The environmental parameter can be one or more of temperature, pH, or presence of a nuclease. Embodiments also include one or more non-transitory, computer-readable medium having computer executable instructions that implement the foregoing computer-implemented methods.

One such method includes the steps of receiving a plurality of nucleotides organized by their position defining a polynucleotide sequence; and applying a deep learning prediction model to the plurality of ribonucleotides to determine a specific property of each nucleotide and the polynucleotide sequence, wherein the deep learning prediction model includes a convolutional neural network and self-attention mechanism to capture local and global dependencies of each nucleotide and the polynucleotide sequence. In an embodiment, properties of single nucleotides are aggregate locally using 1D convolutions before self-attention mechanism was used to obtain a nucleotide to nucleotide attention map. The convolutional neural network can include a 1-D convolution operation or a 2-D convolution operation or a combination of both. The deep learning prediction model can be trained in one or more of a supervised, unsupervised, and semi-supervised learning environments. In an embodiment, the deep learning prediction model receives information from biophysical models. These models include data regarding secondary structures that stabilize a polynucleotide sequence.

Provided here are computer-implemented systems and methods for evaluating structure and stability of polynucleotides to develop a RNA sequence directed to expression of one or more immunogenic proteins of a viral envelope.

The aspects and embodiments described herein can include non-transitory machine readable media that store executable computer program instructions that when executed can cause one or more data processing systems to perform the methods described herein when the computer program instructions are executed by the one or more data processing systems. The instructions can be stored in nonvolatile memory such as flash memory or dynamic random access memory which is volatile or other forms of memory. The above summary does not include an exhaustive list of all embodiments in this disclosure. All systems and methods can be practiced from all suitable combinations of the various aspects and embodiments summarized above, and also those disclosed in the Detailed Description below.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIGS. 1A-1C are diagrammatic representations of the Nucleic Transformer architecture, according to an embodiment. FIG. 1A is a diagrammatic representation of the Nucleic Transformer architecture that combines convolutions and self-attention to capture both local and global dependencies. FIGS. 1B and 1C are schematic representations of the Nucleic Transformer model that can be trained in unsupervised (FIG. 1B), supervised (FIG. 1B), and semi-supervised fashion (FIG. 1C), according to embodiments.

FIG. 2A is a visualization of an attention weight right before outputting a prediction, according to an embodiment. The attention weight heavily favors TTATTAT, which is one mutation away from TATAAT, the consensus promoter motif. FIG. 2B is a graphical representation of the most important 7-mers in promoter classification based on analysis of attention weights, according to an embodiment. FIG. 2C is a graphical representation of the test set AUC across 918 classes with positive samples in predicting effects of non-coding variants, according to an embodiment. FIG. 2D is a graphical representation of the median AUCs in predictions of the transcription factors (TF) binding sites, DNase I sensitivity (DHS), and histone mark (HM) profiles, according to an embodiment. FIG. 2E is a set of box plots of the AUCs of all chromatin features when the Nucleic Transformer models with the same architecture were trained on 200 bp, 500 bp, and 1000 bp input sequences respectively. FIG. 2F is a graphical representation of the comparison of test set performance between the Nucleic Transformer method and the ViraMiner method, according to an embodiment. FIG. 2G is a graphical representation of the ROC AUC curve of ViraMiner versus the Nucleic Transformer method (best end-to-end models), according to an embodiment. FIG. 2H is a graphical representation of the precision-recall curve of ViraMiner vs versus the Nucleic Transformer method (best end-to-end models), according to an embodiment.

FIG. 3A is a visualization of the BPP+distance matrix, attention weights of a non-pretrained Nucleic Transformer, and attention weights of a pretrained Nucleic Transformer. FIG. 3B is a diagrammatic representation of the Nucleic Transformer stack that receives input information from biophysical models, according to an embodiment.

FIGS. 4A-4C is the visualization of BPP+distance matrix (FIG. 4A), attention weights of a non-pretrained Nucleic Transformer (FIG. 4B), and attention weights of a pretrained Nucleic Transformer model (FIG. 4C), respectively, according to an embodiment. FIG. 4D is a graphical representation of the performance of the pretrained and non-pretrained models trained during the OpenVaccine competition and post competition experiments, according to an embodiment. FIGS. 4E-4G are R² score plots of the predictions versus the ground truth of three properties (reactivity and degradation at pH 10 and at 50° C.) of RNA as best ensemble of semi-supervised learning results on the private test set during the OpenVaccine competition, according to an embodiment. FIGS. 4H-4M are R² score plots of the predictions versus the ground truth of three properties (reactivity and degradation at pH 10 and at 50° C.) of RNA on the private test set during the OpenVaccine competition in the other two environments: supervised only (FIGS. 4H-4J) and unsupervised (FIGS. 4K-4M). FIG. 4N is a graphical representation of the half-life correlation of Nucleic Transformer predictions compared to previous best methods, according to an embodiment. FIG. 4O is a graphical representation of the Leave-one-feature-out (LOFO) feature importance on the OpenVaccine dataset, according to an embodiment.

FIG. 5 is graphical representation of the accuracy versus the parameter k of with/without transformer encoder, and with LSTM encoder, according to an embodiment.

FIG. 6 is graphical representation of the cross validation accuracy versus number of random mutations during the training phase, according to an embodiment.

FIG. 7 is graphical representation of the percentage of the one million randomly generated sequences classified as promoters/non-promoters by the Nucleic Transformer method, according to an embodiment.

FIG. 8 is a graphical representation of the effect of the number of transformer encoder layers on test AUC performance, according to an embodiment.

FIG. 9 is a graphical representation of the effect of the width of transformer encoder layers on test AUC performance, according to an embodiment.

FIG. 10 is a t-distributed stochastic neighbor embedding (t-SNE) plot of RNA sequences in the training set, test set, and randomly generated set, according to an embodiment.

FIG. 11 is a graphical representation of a test set performance of OpenVaccine challenge using different pretraining procedures, according to an embodiment.

FIG. 12 is a graphical representation of a Semi-supervised learning with test sequences compared with random sequences, according to an embodiment.

### DETAILED DESCRIPTION

Deep learning is a subclass of machine learning that uses many layers to progressively extract higher-level features from the raw input, and has gained popularity due to its high performance in variety of fields ranging from image and speech recognition, natural language processing and computational biology. Recent advancements in deep learning have allowed researchers to use computational methods to efficiently predict the function, origin, and properties of DNA/RNA sequences from large datasets. The sequential nature of DNA/RNA has made recurrent neural networks (RNN) (such as long short-term memory (LSTM) and gated recurrent unit (GRU)) appealing for models dealing with such data. However, the sequential computation is difficult to parallelize and objects at long distances suffer from vanishing gradients. Transformers, on the other hand, is a recently proposed architecture that solely relies on attention mechanisms that can model dependencies regardless of the distance in the input or output sequences. Transformers have been adopted in many natural language processing tasks, but not in biological systems such as DNA/RNA sequences.

Reference in the specification to “one embodiment” or “an embodiment” means that a particular feature, structure, or characteristic described in conjunction with the embodiment can be included in at least one embodiment. The appearances of the phrase “in one embodiment” in various places in the specification do not necessarily all refer to the same embodiment. The processes depicted in the figures that follow are performed by processing logic that comprises hardware (e.g., circuitry, dedicated logic, etc.), software, or a combination of both. Although the processes are described below in terms of some sequential operations, it should be appreciated that some of the operations described may be performed in a different order. Moreover, some operations may be performed in parallel rather than sequentially. Numerous specific details are described to provide a thorough understanding of various embodiments. However, in certain instances, well-known or conventional details are not described in order to provide a concise discussion of embodiments.

As used herein, the term “polynucleotide” (also “nucleic acid”) refers to a macromolecule made up of nucleotides of any length, either deoxyribonucleotides or ribonucleotides or analogs thereof. The sequence of a polynucleotide can be composed of one or more of the four nucleotide bases—adenine (A), cytosine (C), guanine (G), thymine (T); with uracil (U) for thymine when the polynucleotide is RNA. A polynucleotide can include a gene or gene fragment, exons, introns, coding or non-coding DNA or combinations, coding or non-coding RNA or combinations, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. The nucleic acids can be double- or single-stranded and may contain double- and single-stranded fragments at the same time. The term encompasses nucleic acids containing known nucleotide analogs or modified backbone residues or linkages, which are synthetic, naturally occurring, and non-naturally occurring. Examples of such analogs include, without limitation, phosphorothioates, phosphoramidates, methylphosphonates, chiral-methyl phosphonates, 2-O-methyl ribonucleotides, peptide-nucleic acids (PNAs), and locked nucleic acid (LNA).

Disclosed here is a convolution and transformer based deep learning platform directed to solving polynucleotide tasks, such as DNA sequence identification, RNA stability predictions, RNA decay and structure relationships, next generation mRNA therapy development, and extraction of information from unknown DNA and RNA sequences. A computer-implemented method termed “Nucleic Transformer” was developed that utilizes convolution and self-attention to capture both local and global dependencies. This method enables high accuracy and provides interpretability to the predicted results for a variety of nucleic acid tasks. The Nucleic Transformer method formulates DNA understanding as natural language processing tasks, and RNA degradation as molecular prediction tasks. In an embodiment, the Nucleic Transformer method requires little domain knowledge and thus can provide both improved prediction accuracy and enhanced interpretability on various polynucleotide development processes, including E. coli promoter/non-promoter classification, viral/non-viral sequence classification, and RNA stability evaluation.

In an embodiment, the Nucleic Transformer method was used to classify a short piece of DNA sequence as either a bacterial promoter sequence or a non-promoter sequence. In an embodiment, the Nucleic Transformer was used to classify a short piece of DNA sequence as either a eukaryotic promoter sequence or non-promoter sequence. Accurate identification of promoter is in high demand as it is critical for gene expression regulation. Molecular techniques that place all the sequences that need to be identified upstream of a reporter gene to determine promoter characterization are resource-extensive. Traditional classification approaches such as NCBI BLAST that depend on alignment-based classification, in which sequences are aligned to known genomes from public databases, have limited application for novel viral sequences that are highly divergent from the existing database. As the Nucleic Transformer captured both local and global dependencies, it provided promoter identification of both viral and non-viral sequences with improved accuracy as compared with a previous computational models.

In an embodiment, the Nucleic Transformer was used to predict effects of noncoding variants from 1000 bp fragments and was demonstrated to have greater accuracy than the DeepSEA model (available at https://hb.flatironinstitute.org/deepsea/) for this function. In an embodiment, the Nucleic Transformer was used for enhancer predictions. In an embodiment, the Nucleic Transformer was used for classification of nucleotide sequences as viral or non-viral sequences. For example, the Nucleic Transformer was tested on DNA sequences (300 bp) for classification of viral and non-viral sequences. The Nucleic Transformer predicts both viral and non-viral sequences with better accuracy as compared to ViraMiner (another computational model described in https://journals.plos.org/plosone/article?id=10.1371/journal.pone.0222271).

In an embodiment, the Nucleic Transformer was used for prediction of RNA degradation rates at each position of a given sequence. RNA stability is of particular importance in the development of RNA-based diagnostics and therapeutics. Messenger RNA (mRNA) vaccines were the fastest developed candidates for COVID-19, but they can degrade easily under ambient conditions. Accurate computational analysis of the stability of RNA sequences helps the selection and optimization of potential mRNA vaccine candidates. The Nucleic Transformer method has been developed to address the stability of RNA sequences with different machine learning approaches, including supervised-learning, supervised learning with unsupervised learning, and semi-supervised learning.

The Nucleic Transformer method is based on an effective architecture for predicting effects of non-coding variants, classifying promoters, viral genome and enhancers, as well as predicting degradation properties of RNA. In an embodiment, the Nucleic Transformer effectively learns from 1000 bp context and shows improved performance over previously known DeepSea model. Further, the Nucleic Transformer architecture outperforms other deep learning/non deep learning methods that require hand-crafted features in promoter classification, while also providing interpretability and being capable of extracting promoter motifs directly from learned attention. Next, the Nucleic Transformer method provides better performance than previous best model at enhancer predictions. Although always trained end to end, the Nucleic Transformer method has better accuracy in classifying viral genomes compared to previous models such as ViraMiner, which requires sophisticated multi-stage training and ensembling. The Nucleic Transformer method was used in the recent OpenVaccine challenge and placed 7th out of 1636 teams of top machine learning experts from all over the globe. With semi-supervised learning, the Nucleic Transformer outperforms even the top solution in the OpenVaccine challenge by a considerable margin; Nucleic Transformer method also predicts half-lives of mRNA sequences with better correlation than previous known methods. Self-attention and convolution are a powerful combination for genomics tasks, enabling learning both global and local dependencies effectively.

Although classification of promoters and viral genomes have been well studied in the literature, there is no precedent on predictions of mRNA degradation properties per nucleotide. To address the COVID-19 epidemic, mRNA 228 vaccines have emerged as a fast and effective solutions, with companies like Pfizer and Moderna rolling out mRNA vaccines at unprecedented speeds. However, storage and transport remain a challenge with fragile mRNA vaccines (Pfizer-BioNtech's vaccine has to be stored as −80° C. and Moderna's at −20° C.). One strategy to reduce mRNA hydrolysis is to redesign RNAs to code for the same proteins but form double-stranded regions, which are protected from these degradative processes. The Nucleic Transformer method provides guidance and trained models that act as a screening tool in development of more stable mRNA vaccines. The mRNA-based vaccines and therapeutics have many applications towards infectious diseases and cancers. The Nucleic Transformer method aids in the design of more stable mRNA vaccines that can withstand harsher conditions than current ones.

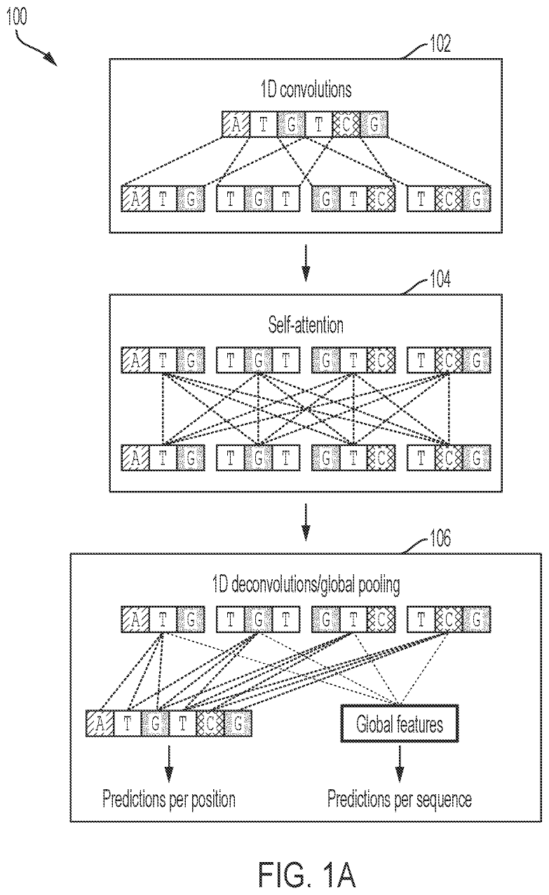

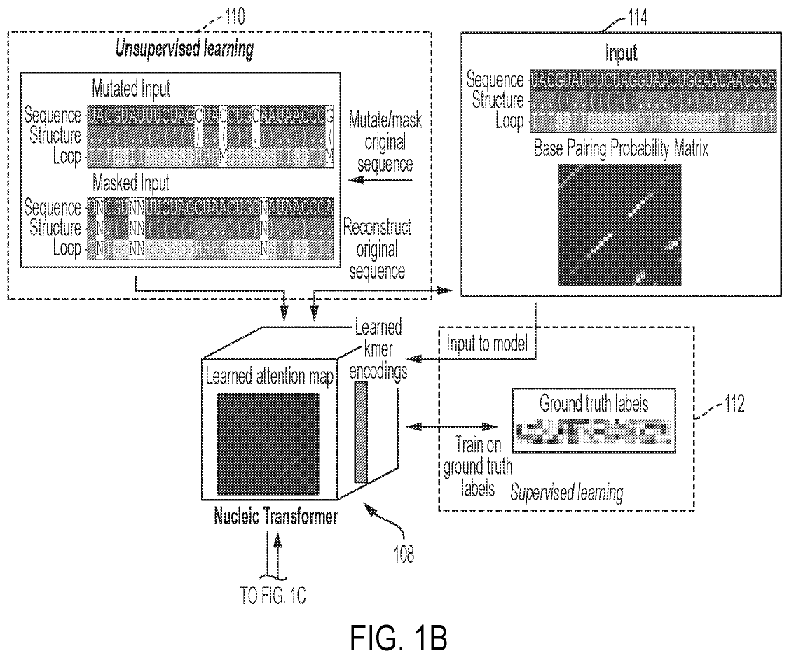

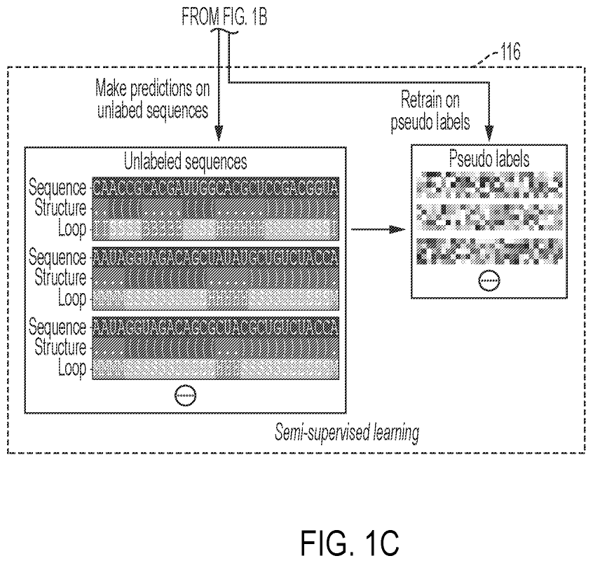

FIGS. 1A-1C are diagrammatic representations of the Nucleic Transformer architecture, according to an embodiment. FIG. 1A is a diagrammatic representation of the Nucleic Transformer architecture that combines convolutions and self-attention to capture both local and global dependencies. The Nucleic Transformer method architecture combines convolutions and self-attention to capture both local and global dependencies (FIG. 1A), and can be trained in both supervised and unsupervised fashion to make predictions per sequence and per nucleotide. Its flexible nature also allows injection of information from biophysical models. The self-attention mechanism explicitly models pairwise interactions and provides interpretability that allows more informed decision making.

In FIG. 1A, the method 100 includes the step 102 at which the DNA sequences are transformed into k-mers with 1D convolutions, a deep learning operation that allows for efficient and effective motif recognition. The extraction of k-mers from a DNA sequence can be considered a sliding window of size k taking snapshots of the sequence while moving one position at a time from one end of the sequence to the other, which is conceptually identical to the convolution operation used in deep learning. The self-attention function essentially computes a pairwise interaction matrix relating every k-mer to every k-mer (including self to self interaction) and computes a weighted sum of the values. Although effective at motif recognition, convolution on its own cannot capture long-range dependencies that are prevalent in DNA/RNA. Other works in the literature have applied recurrent neural networks such as LSTM (Long short term memory) networks, but LSTM's sequential nature means that it is still insufficient at modeling relationships at long distances. To enable the neural network here to learn long-range dependencies at any distance, at step 104, transformer encoder layers with self-attention were used following the 1D convolution layers. Self-attention networks, which process the entire sequence altogether, allow for faster computation compared to other sequence processing methods such as recurrent neural networks and also perform better at capturing long-range dependencies. Following the transformer encoder layers, one output layer (global pooling/deconvolution followed by a linear layer) was used at step 106 right before making predictions appropriate for the task. Combining 1D convolutions and self-attention makes the neural networks adept at modeling both local motifs and long-range dependencies, two key aspects of modeling DNA/RNA. Additionally, the neural network architecture 108 learnt with different learning approaches depending on the task at hand. FIGS. 1B and 1C are schematic representations of the Nucleic Transformer model that can be trained in unsupervised (FIG. 1B), supervised (FIG. 1B), and semi-supervised fashion (FIG. 1C), according to embodiments. The three learning methods are highlighted in FIGS. 1B and 1C and are: supervised learning 112 (where the neural network learns from labeled input), unsupervised learning 110 (where the neural network learns directly from unlabeled input), and semi-supervised learning 116 in FIG. 1C (where the training combines a small amount of labeled data and a larger amount of unlabeled data). Multitasking learning: during pretraining, mutated/masked sequence, structure, and predicted loop type, along with a base pairing probability matrix, are inputted as 114 into the Nucleic Transformer architecture 108 and then the Nucleic Transformer method is trained with crossentropy loss to retrieve the correct sequence, structure, and predicted loop type simultaneously at each position of the RNA sequence. During training on ground truth labels and pseudo labels, the Nucleic Transformer model was trained to predict five different degradation properties simultaneously at each measured position of the RNA sequence.

Unsupervised learning: Although pretraining was not used for the DNA tasks, all available sequences in the OpenVaccine challenge dataset were used to pretrain the network on randomly mutated and masked (with NULL token) sequence retrieval loss (basically softmax to retrieve correct nucleotide/structure/loop). During pretraining, the Nucleic Transformer model learns the rules of RNA structure, guided by biophysical knowledge provided by biophysical models.

Supervised/semi-supervised learning: During supervised learning, the Nucleic Transformer model is trained on target values of classification classes or RNA degradation properties. Following RNA supervised learning, the Nucleic Transformer model was retrained in semi-supervised fashion on pseudo labels generated by an ensemble of the Nucleic Transformer models with different depths. Similar to previous work with semi-supervised learning, the models were retrained first using pseudo labels at a flat learning rate and then fine-tuned with ground truth labels in the training set with cosine anneal schedule.

Usage of biophysical models. Secondary structures predicted by an ensemble of biophysical models were used including RNA soft, rnastructure, CONTRAfold, Eternafold, nupack, and Vienna. For each sequence, secondary structure predictions were generated at 37 and 50 Celsius, as two of the scored degradation properties were measured at different temperatures. Although predictions for a degradation property at pH10 were needed, none of the biophysical models used could generate predictions at different pH's. With 6 packages, 12 secondary structure predictions for each sequence were generated. During training, one of the 12 secondary structure predictions was randomly selected for each sample during a forward and backward propagation pass. During validation and testing, the averaged predictions used all 12 secondary structure predictions.

Promoter/non-promoter classification. In an embodiment, the Nucleic Transformer was used to classify a short piece of DNA sequence (81 bp) as either a bacterial promoter sequence (e.g., an E. coli promoter sequence) or a non-promoter sequence. In an embodiment, the Nucleic Transformer was used to classify a short piece of DNA sequence as either a eukaryotic promoter sequence or non-promoter sequence.

The performance of the Nucleic Transformer method with the best hyperparameters on E. coli promoter classification was compared to the performance of other methods in the literature. Then, the effects of certain hyperparameters on the accuracy of the results were explored, followed by visualization and analysis of attention weights. The Nucleic Transformer method outperformed previous results in the literature in E. Coli promoter classification (Table 1). Compared to the non-deep learning approaches which use sophisticated hand crafted features, the Nucleic Transformer method leads in accuracy by at least 1.7% or more. A more recent model, iPromoter-BnCNN, which also employs structural property properties of DNA such as stability, rigidity, and curvature, is similar in performance, although the Nucleic Transformer method makes predictions from sequence information directly. The best results were obtained using k=7, nₘᵤₜₑ=15, six transformer encoder layers, d_{model}=256, n_{head}=8, and batch size is 24.

TABLE 1

| Name of Model | Accuracy | MCC | Sensitivity | Specificity |
|---|---|---|---|---|
| Nucleic Transformer | 0.8831 | 0.7665 | 0.8935 | 0.8727 |
| iPromoter-BnCNN | 0.882 | 0.763 | 0.883 | 0.88 |
| MUL TiPly | 0.8668 | 0.7224 | 0.8656 | 0.8668 |
| iPromoter-2L2.0 | 0.8498 | 0.6998 | 0.8413 | 0.8584 |
| iPromoter-2L1.0 | 0.8168 | 0.6343 | 0.792 | 0.8416 |

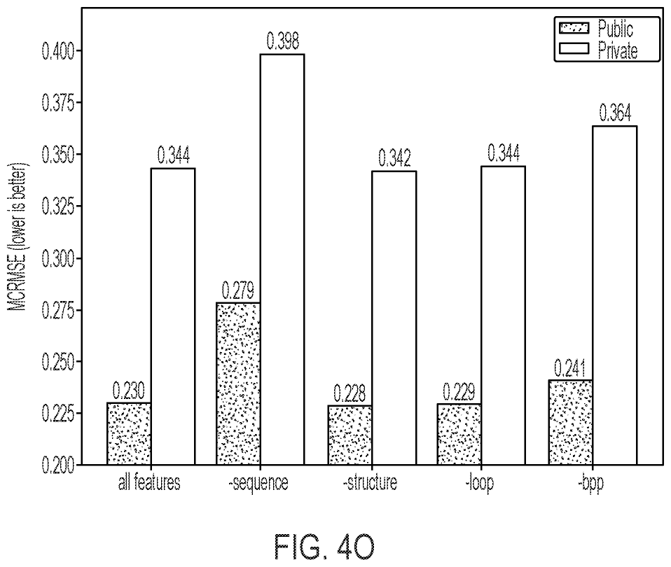

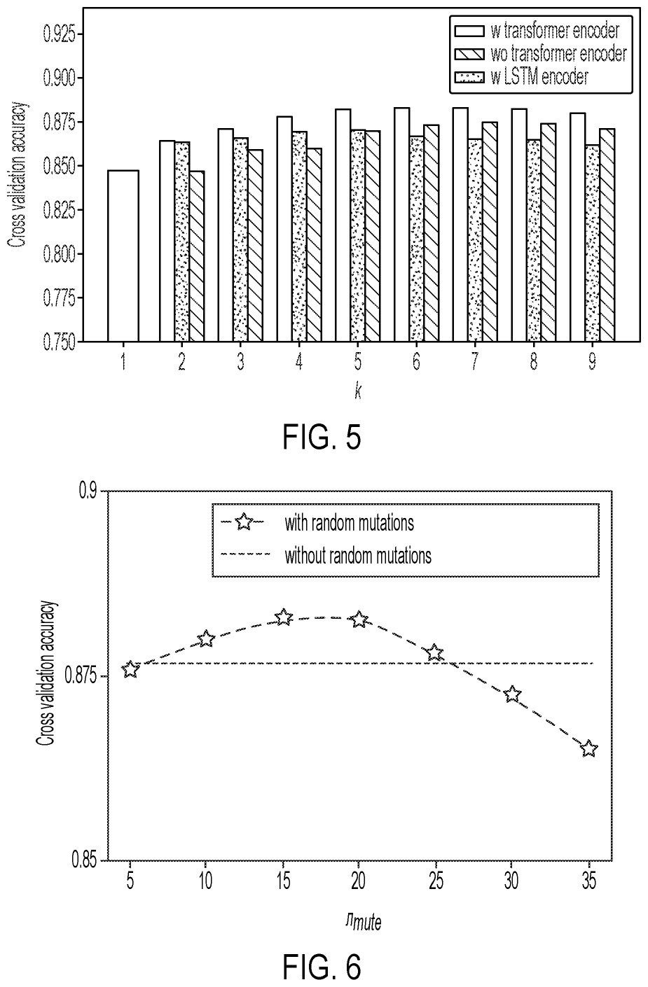

Further, to compare the transformer encoder of the Nucleic Transformer method with LSTM, the transformer encoder with a Bidirectional LSTM was swapped out while keeping all other conditions the same. The transformer encoder consistently outperforms LSTM 96 on all k values (FIG. 5). On the independent test set (Table 2), which includes recently released experimentally verified promoter samples, the Nucleic Transformer model was more accurate than MULTiPly, iPromoter-2L, and iPromoter-BnCNN models.

TABLE 2
K-mers of different lengths

| Name of Model | True Positives | False Negatives |
|---|---|---|
| Nucleic Transformer | 246 | 10 |
| iPromoter-BnCNN | 245 | 11 |
| MULTiPly | 238 | 18 |
| iPromoter-2L | 238 | 18 |

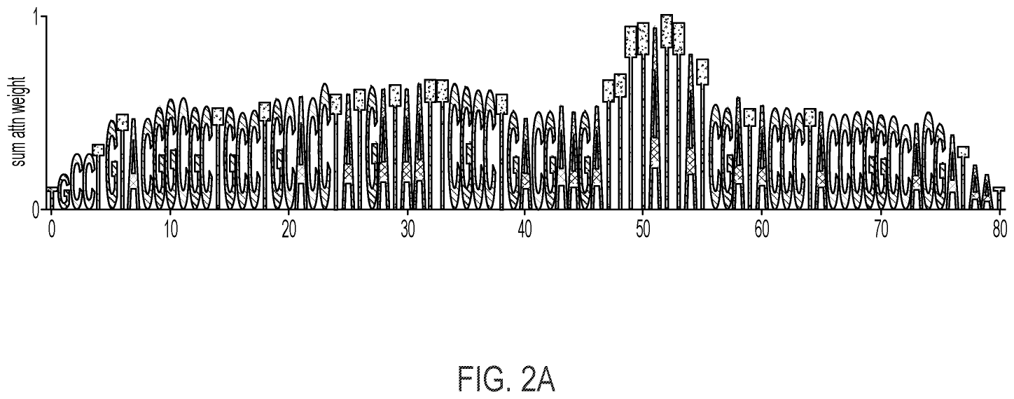

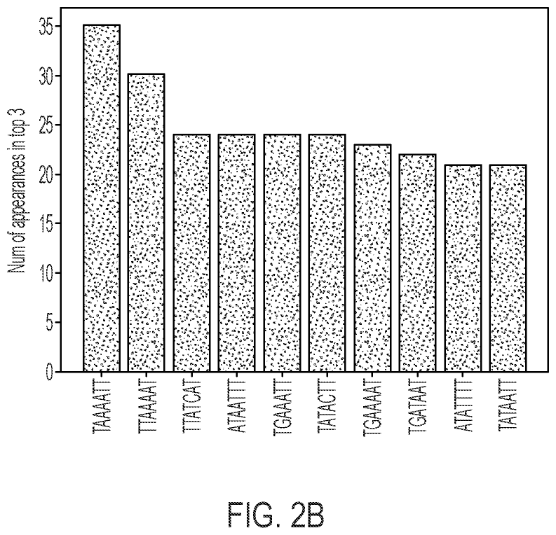

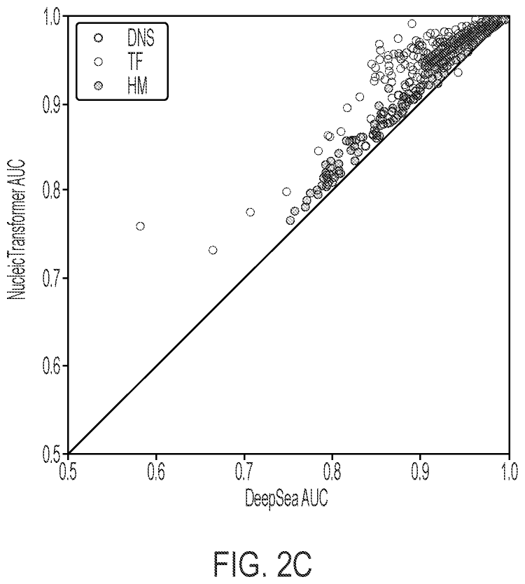

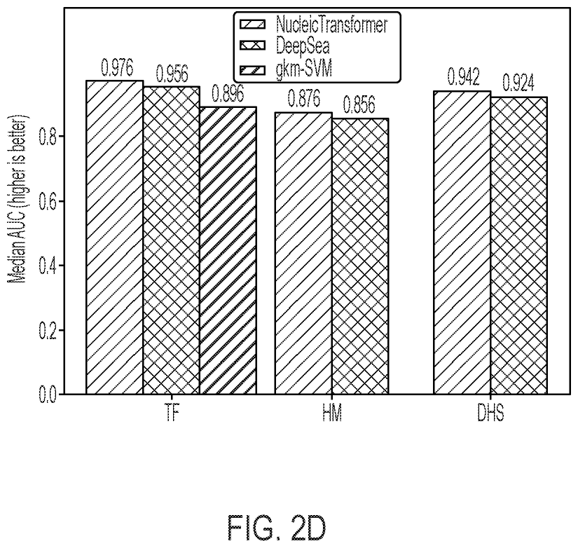

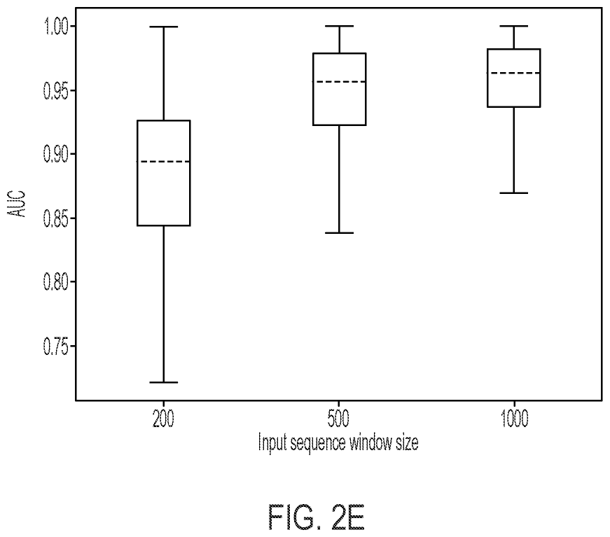

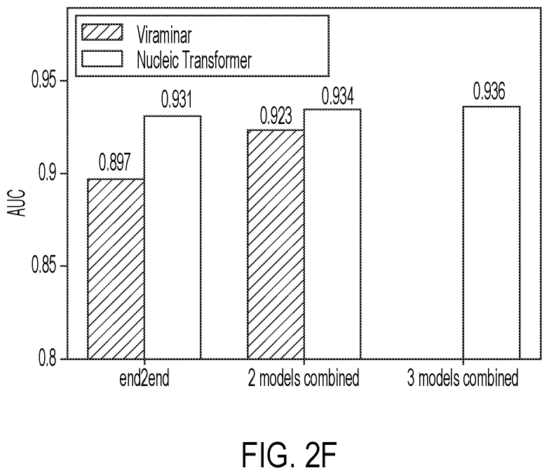

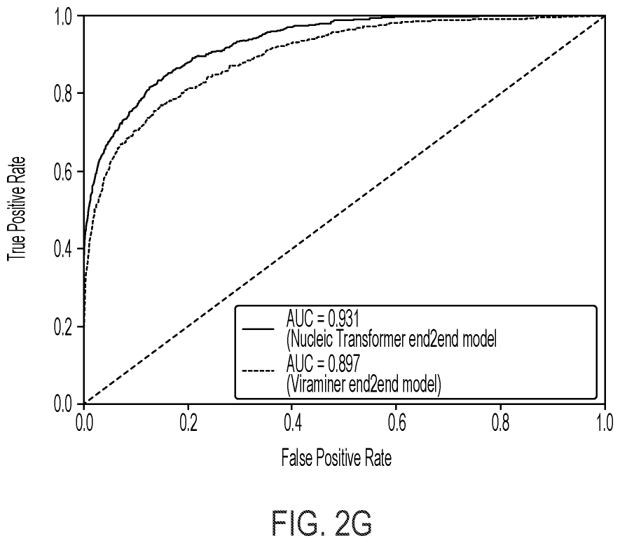

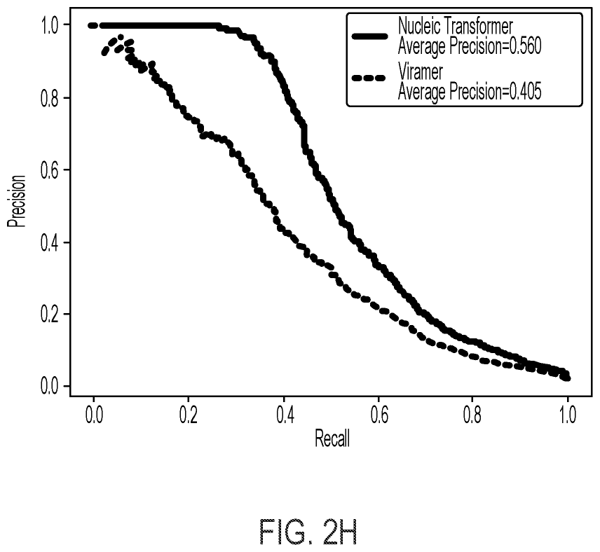

By visualizing the attention weight matrix, the Nucleic Transformer often focused on k-mers that closely 100 resemble consensus promoter motifs (FIG. 2A). Motifs that the Nucleic Transformer considered as the 101 most characteristic of promoters were also extracted (FIG. 2B). The k-mers that frequently appear in top 3 resemble the consensus promoter motif TATAAT. In fact, one of the 10 most frequently 7-mers have the exact consensus motif TATAAT, while 6 others contain the motif TAAAAT, TATCAT, ATAAT, TATACT, GATAAT, all of which are one mutation away from the consensus motif TATAAT. These results demonstrate that the Nucleic Transformer can be interpreted to understand biological mechanisms of DNA.

Aside from E. coli promoters, the performance of the Nucleic Transformer method to classify mouse and human promoters was evaluated. Previously available DeePromoter can accurately distinguish between mouse and human (TATA/non-TATA) promoters and promoter sequences with random mutations of segments. The Nucleic Transformer method includes a different route in constructing the negative dataset by using the flanking regions of promoter sequences, which results in roughly a 2:1 class (non-promoters to promoters) imbalance. While the Nucleic Transformer outperforms DeePromoter only by a small margin on classification of TATA promoters of human and mouse, on the more difficult tasks of classifying non-TATA promoters, the Nucleic Transformer leads in performance by large margins (0.22/0.35 MCC) in human/mouse non-TATA promoters (Table 3).

TABLE 3
Performance of Nucleic Transformer across human
and mouse species compared to previous results.

| Organism | Model | Accuracy | Precision | Recall | F1 | MCC |
|---|---|---|---|---|---|---|
| Human TATA | DesPromoter | 0.9732 | 0.943 | 0.9785 | 0.9604 | 0.94 |
| | NucleicTransformer | 0.9775 | 0.9621 | 0.9706 | 0.9663 | 0.9494 |
| Human non-TATA | DeePromoter | 0.8373 | 0.8575 | 0.9592 | 0.9055 | 0.363 |
| | NucleicTransformer | 0.8829 | 0.9064 | 0.9544 | 0.9298 | 0.585 |
| Mouse TATA | DeePromoter | 0.9751 | 0.9513 | 0.9779 | 0.9644 | 0.9456 |
| | NucleicTransformer | 0.9776 | 0.958 | 0.9779 | 0.9678 | 0.9508 |
| Mouse non-TATA | DeePromoter | 0.8342 | 0.8518 | 0.954 | 0.9 | 0.4497 |
| | NucleicTransformer | 0.9015 | 0.9271 | 0.9487 | 0.9378 | 0.7037 |

To experiment with k-mers of different lengths, first a baseline was set with no k-mer aggregation, where only single nucleotides with positional encoding were inputted into the transformer. Then k was incrementally increased, and its effect on cross validation accuracy was evaluated (FIG. 5). Additionally, experiments were conducted without the transformer encoder to evaluate the improvement provided by the transformer encoder, in which case the k-mer aggregation layer was followed by a maxpooling and fully connected layer. FIG. 5 is graphical representation of the accuracy vs the parameter k of with/without transformer encoder, and with LSTM encoder. This embodiment used nₘᵤₜₑ=15, six transformer encoder layers, d_{model}=256, n_{head}=8, and batch size is 24.

The model's performance improved initially upon using longer k-mers, and then saturated to a point where using longer k-mers no longer increased performance. Aggregating larger k-mers (k=8, 9) actually led to a decrease in performance, likely due to over-fitting resulting from large convolution kernels. Further, the transformer encoder consistently provided a significant boost in accuracy compared to the counterpart without transformer encoder.

Number of random mutations during training. There is an optimal value for the number of random mutations during training (FIG. 6). Here six transformer encoder layers were used, d_{model}=256, n_{head}=8, and batch size is 24. FIG. 6 is graphical representation of the cross validation accuracy vs number of random mutations during the training phase. For experiments without the transformer encoder, the transformer encoder was removed while keeping everything else the same. The model tended to overfit to the training data when no random mutations are added during training. Without mutations, the model quickly converges to 1.0 accuracy and close to 0 loss on the training set, indicating that the model has simply memorized the training data. Additionally, too many mutations lead to underfitting, as there is too much random noise during training leading to poor convergence.

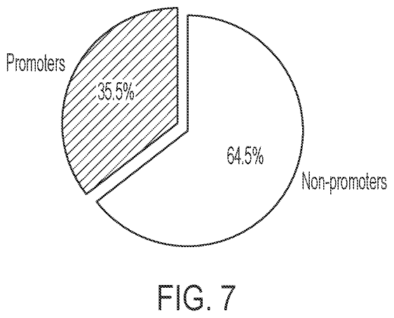

Model behavior on randomly generated DNA sequences. It has been experimentally shown that approximately 10% random sequences can function as promoters. Here, one million randomly generated DNA sequences of length 81 were screened, and classified using the model described here (FIG. 7). Despite a balanced distribution of positive and negative examples in the promoter/non-promoter dataset, the Nucleic Transformer model recognized approximately 65% of random DNA sequences as non-promoters. FIG. 7 is graphical representation of the percentage of the one million randomly generated sequences classified as promoters/non-promoters by the Nucleic Transformer method. The Nucleic Transformer model has a mechanism to guard against random sequences and hence is able to generalize well.

Comparison with ViraMiner. When trained end to end, the Nucleic Transformer method significantly outperforms the ViraMiner counterpart by 3.4% AUC score. When trained with a two-stage training process combining two branches with different hyperparameters and pooling schemes, the ViraMiner model performed significantly better compared to its end to end counterpart, but the Nucleic Transformer model still leads in accuracy even with just one end to end model (k=13). Averaging results from a few machine learning models trained with slightly different hyperparameters usually leads to better performance, which explains the success of the ViraMiner model's strategy to combine two branches with different setups. For better comparison, the Nucleic Transformer model was trained two and three times with different ks and averaged the test set predictions. The AUC improved upon by 0.3% upon averaging 2 models (k=11, 13), but with 3 models averaged (k=11, 13, 15), the AUC only improved slightly by 0.2% in this embodiment, nₘᵤₜₑ=40, six 364 transformer encoder layers, d_{model}=512, and n_{head}=8.

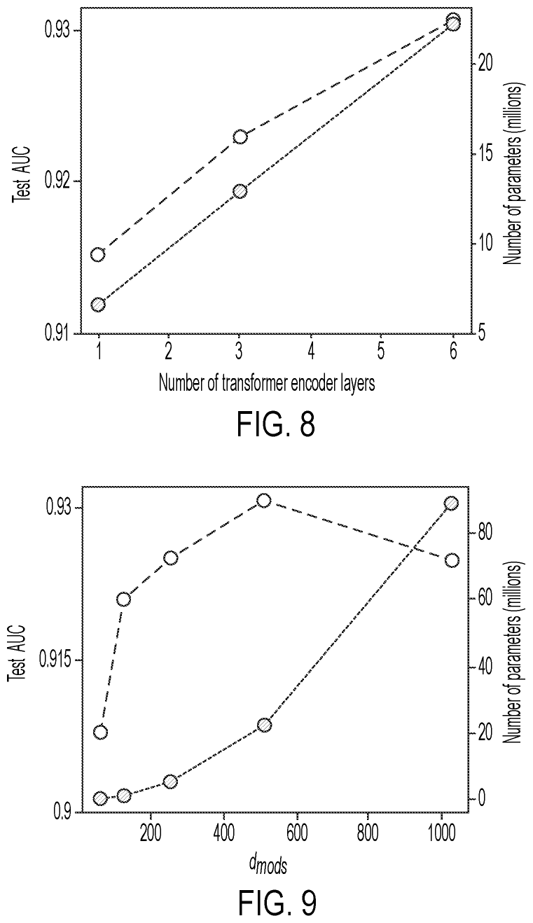

Model width and depth. Previous works utilizing genomics used relatively shallow networks and did not explore the effects of varying the depth or width of neural networks. When hypertuning the Nucleic Transformer model, increasing the depth and width of the transformer encoder generally led to better performance on the viral/non-viral dataset (FIGS. 8 and 9), although increasing the model width to 1024 actually led to worse results, mostly due to overfitting. FIG. 8 is a graphical representation of the effect of the number of transformer encoder layers on test AUC performance. FIG. 9 is a graphical representation of the effect of the width of transformer encoder layers on test AUC performance. Notably, increasing model depth beyond six transformer encoder layers actually led to diverged training on the viral genome dataset, probably because of the huge class imbalance. Increasing the depth and width lead to better performance. An embodiment of the model includes only an increase in the depth of the transformer encoder, because the number of parameters increase quadratically with the width of the transformer encoder.

Nucleic Transformer Outperforms DeepSea in Predicting Effects of Noncoding Variants.

Identifying functional effects of noncoding variants is a challenging and important task, a noncoding genomic variations make up the majority of disease and other trait-associated single-nucleotide polymorphisms (SNPs). DeepSea's performance with a purely convolution based deep learning architecture significantly outperforms gkm-SVM, a classical machining model that utilizes gapped k-mer features. But, the Nucleic Transformer method outperforms DeepSea. The Nucleic Transformer method includes training models with 919 binary classifiers that were trained on the dataset used to train DeepSea. The Nucleic Transformer method demonstrates better AUC across almost all the 918 classes with positive samples on the test set (FIG. 2C). In some classes Nucleic Transformer shows an AUC improvement of 5-10%. Comparing the median AUC of predicting transcription factors (TF) binding sites, DNase I sensitivity (DHS), and histone mark (HM) profiles (FIG. 2D), the Nucleic Transformer holds a sizable advantage over DeepSea in predicting effects of noncoding variants. A statistically significant increase in model performance was observed when increasing context length from 200, to 500 and 1000 bp (FIG. 2E and Table 4). The AUCs of all chromatin features were shown with box plots. Increasing context sequence length significantly improved model performance (P-value<4.10e−141 by Wilcoxon signed rank test between any pair of models). These results demonstrate the ability of the Nucleic Transformer method to capture long-range dependencies. Looking at median AUCs of different groups, the most substantial increase in performance in HM predictions was when training with longer context.

TABLE 4
Median AUCs of Nucleic Transformer when
trained with 200, 500, 1000 bp contexts.

| context/median AUC | TF | DNS | HM |
|---|---|---|---|
| 200 | 0.905 | 0.899 | 0.773 |
| 500 | 0.933 | 0.969 | 0.834 |
| 1000 | 0.939 | 0.973 | 0.872 |

Nucleic Transformer Accurately Identifies DNA Enhancers.

While promoters initiate the transcription process, enhancers increase the likelihood of transcription; accurately identifying enhancers is an important task in bioinformatics. Using an enhancer dataset previously used to train the bert-enhancer, the Nucleic Transformer models were trained to classify enhancers and non-enhancers. The Nucleic Transformer method outperforms previous state of the art bert-enhancer by a considerable margin in accuracy and Matthews correlation coefficient (MCC) in both cross validation and independent testing dataset (Table 5 and Table 6).

TABLE 5
Cross validation performance on enhancer prediction.

| Model | Sensitivity | Specificity | Accuracy | MCC |
|---|---|---|---|---|
| fastText | 0.761 | 0.744 | 0.753 | 0.505 |
| Bert-enhancer | 0.795 | 0.73 | 0.762 | 0.525 |
| NucleicTransformer | 0.779 | 0.772 | 0.786 | 0.558 |

TABLE 6
Independent test set performance on enhancer prediction.

| Model | Sensitivity | Specificity | Accuracy | MCC |
|---|---|---|---|---|
| EnhancerPred | 0.735 | 0.745 | 0.74 | 0.48 |
| iEnhancer-2L | 0.71 | 0.785 | 0.7475 | 0.496 |
| iEnhancer-EL | 0.71 | 0.772 | 0.786 | 0.46 |
| Bert-enhancer | 0.8 | 0.712 | 0.756 | 0.514 |
| NucleicTransformer | 0.81 | 0.75 | 0.78 | 0.561 |

Nucleic Transformer Outperforms Previous Models in Viral Genome Classification.

To further demonstrate the effectiveness of the Nucleic Transformer architecture, the Nucleic Transformer model was trained on a viral/non-viral genome dataset previously used to train ViraMiner, a purely convolution based model, and performance of the two models was compared. FIG. 2F is a graphical representation of the comparison of test set performance between the Nucleic Transformer method and the ViraMiner method. When trained end-to-end, the Nucleic Transformer significantly outperforms the ViraMiner counterpart by 3.9% (Area Under the ROC Curve) AUC score (Table 7).

TABLE 7
Statistical test of ViraMiner vs Nucleic Transformer (end-to-
end). Nucleic Transformer showed significant better performance
(Wilcoxon signed-rank test p-value < 0.0051, N = 10)

| | Mean (AUC) | Standard Deviation |
|---|---|---|
| Nucleic Transformer | 0.91921 | 0.006742806866909666 |
| Viraminer | 0.88013 | 0.00876014332708726 |

When trained with a two-stage training process combining two branches with different hyperparameters and pooling schemes, the ViraMiner performs significantly better compared to its end to end counterpart, but the Nucleic Transformer still leads in accuracy even with just one end to end model (k=13). For better comparison, the Nucleic Transformer models were also trained 2 and 3 times with different k's and averaged the test set predictions. The AUC improved upon by 0.3% upon averaging 2 models (k=11, 13), but with 3 models averaged (k=11, 13, 15), the AUC only improved slightly 147 by 0.2%, which is expected as with more models ensembled there is usually diminishing returns. The precision recall curve and ROC curve were compared between the best end-to-end models of ViraMiner and the Nucleic Transformer models. (FIG. 2G, FIG. 2H). These results demonstrate that the Nucleic Transformer holds a clear advantage in end-to-end model performance.

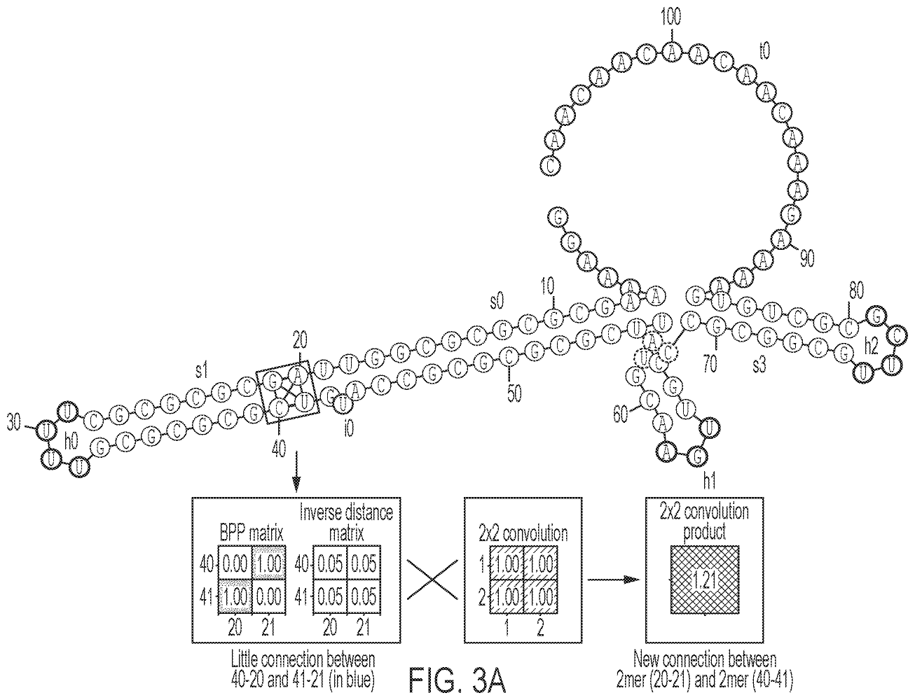

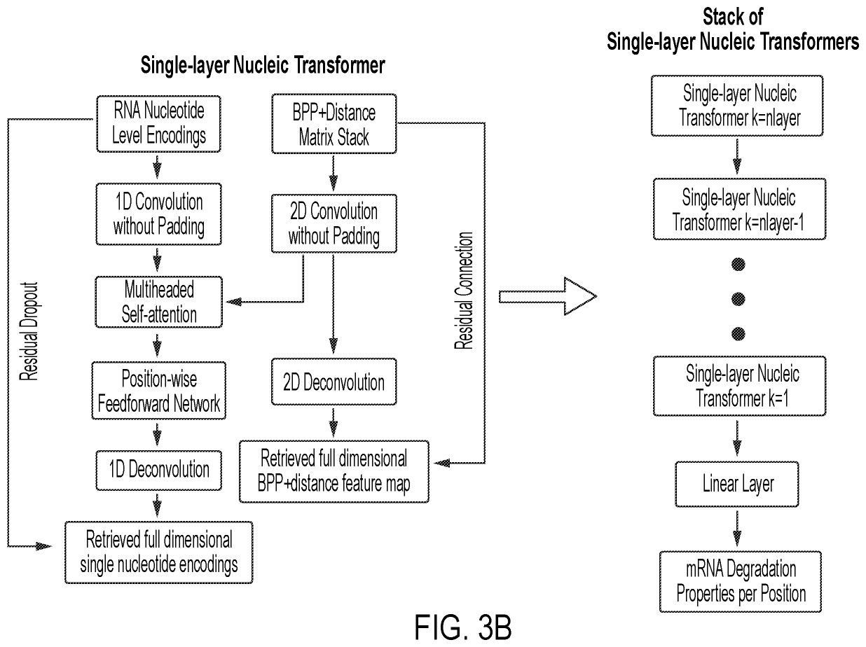

Nucleic Transformer accurately predicts RNA degradation at nucleotide level. RNA vaccines and therapeutics have incredible potentials, but one key limitation is the inherent instability of mRNA. The 21-day OpenVaccine challenge, hosted by Das Lab at Stanford University, sought to understand RNA degradation by rallying the expertise of data scientists from all over the globe. An adaption of the Nucleic Transformer method was used in this challenge with some key modifications. Unlike DNA which is double stranded and relatively stable, RNA is single-stranded and highly promiscuous. While double-stranded DNA forms hydrogen bonds between its complementary bases, single-stranded RNA forms secondary structures by itself, which have been known to stabilize RNA molecules. Therefore, existing biophysical models were used to inject knowledge of RNA secondary structure into the deep learning models to predict RNA degradation. To do so, the base pairing probability map was added following 2D convolutional layers as an additional bias to the self-attention weight in self-attention layers (FIG. 3A), while adding additional embedding layers to represent structure and loop predictions from secondary structure models. The BPP+distance matrix and attention weights are directly visualized using base pairing map produced by RNA folding algorithms and attention weights are learnt from the folding and experimental degradation data. In an embodiment, the attention weights were learned using sequence/structure/loop and BPP+distance matrix as inputs to the neural network, and fitting to the degradation properties as measured in experiments.

The modified Nucleic Transformer architecture used for the RNA task is illustrated in FIG. 3B. First, the single-layer Nucleic Transformer method receives the predicted structure per position, which describes whether a nucleotide is paired or unpaired with another one via hydrogen bonding. Also, the predicted loop type assigned by bpRNA is also inputted into this step of the single-layer Nucleic Transformer method. Together with sequence information, three embedding layers are added to represent sequence, structure, and loop type, and the resulting feature vectors are concatenated and the dimensionality reduced with a linear transformation. Further, a modified version of the base-pairing probability matrix after going through a 2D-convolution layer is directly added into the self-attention function. Afterwards, a position-wise feedforward network is used to transform the hidden representations. The combination of convolution and self-attention cannot produce nucleotide position wise predictions, since it generates kmer encodings instead of single base pair encoding. In order to make predictions per nucleotide position, additional deconvolution layers were introduced to retrieve full dimensional encodings, which allow residual connections of both 1D and 2D encodings before and after the transformer encoder. As a result, both the single nucleotide embeddings and the modified BPP matrix go through deep transforms before outputting predictions. This architecture includes a series of multiple Nucleic Transformers with a single transformer encoder layer followed by a deconvolution layer. Also, when predictions are required at each position of the RNA sequence, it is important for the last transformer encoder layer right before outputting predictions to operate on single nucleotide encodings instead of k-mer encodings. A stack of Nucleic Transformers was constructed with two main hyperparameters k and nlayer set equal to each other. The first single layer Nucleic Transformer has k=nlayer and the size of the convolution kernel was decreased by 1 for the next single layer Nucleic Transformer. Therefore, when the last Nucleic Transformer in the stack is reached, k becomes 1 and the last Nucleic Transformer is simply a transformer encoder layer with an added bias from the BPP feature map.

Interpretability of Self-Attention Helps Decision Making.

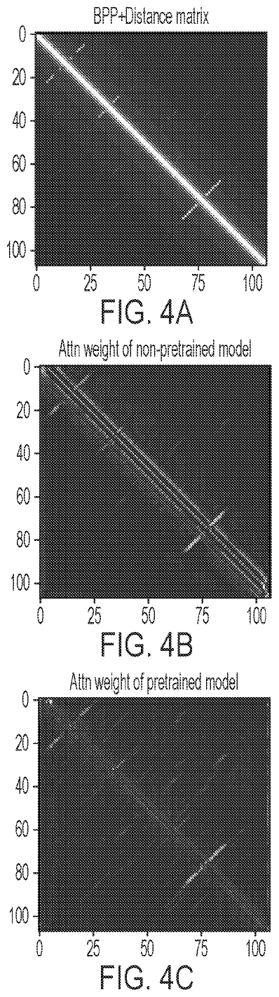

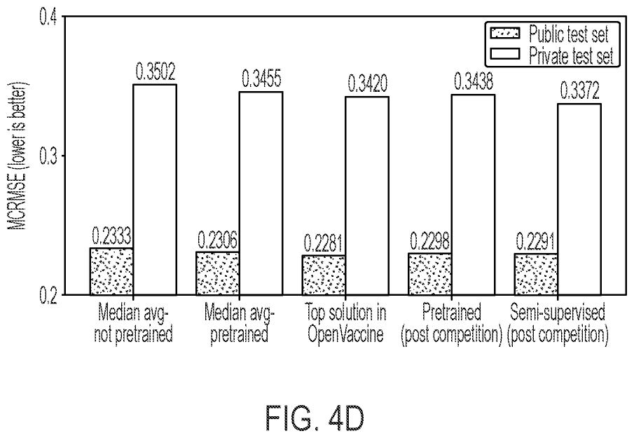

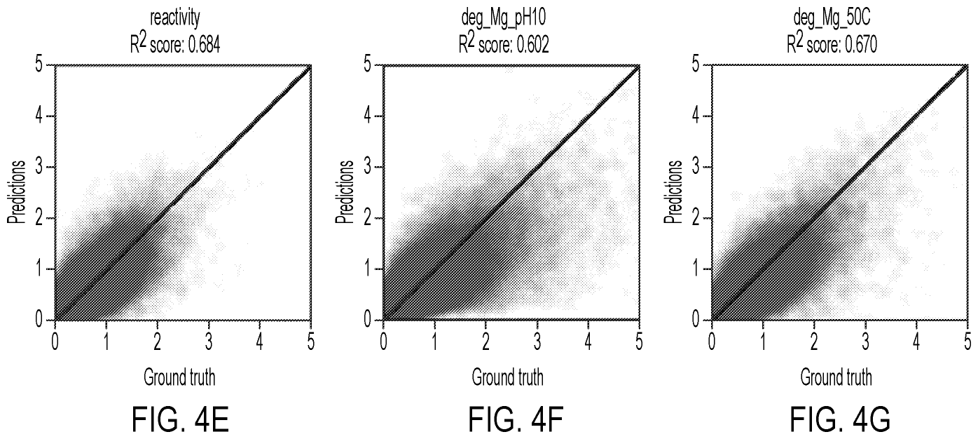

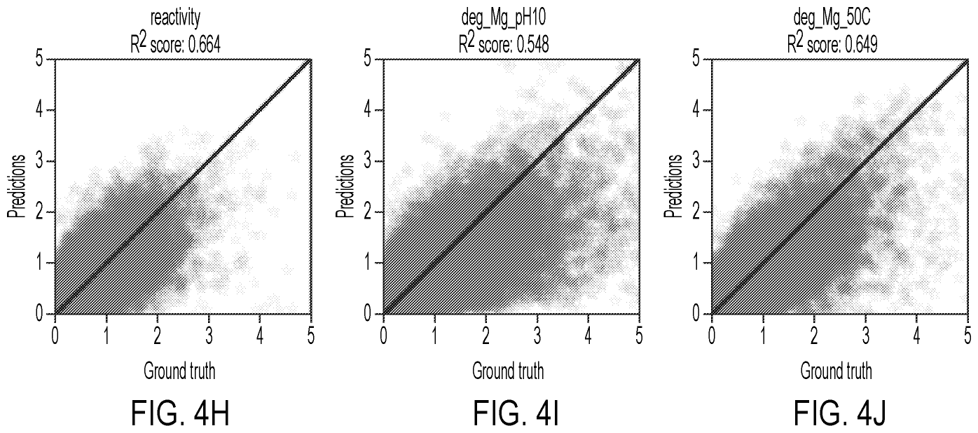

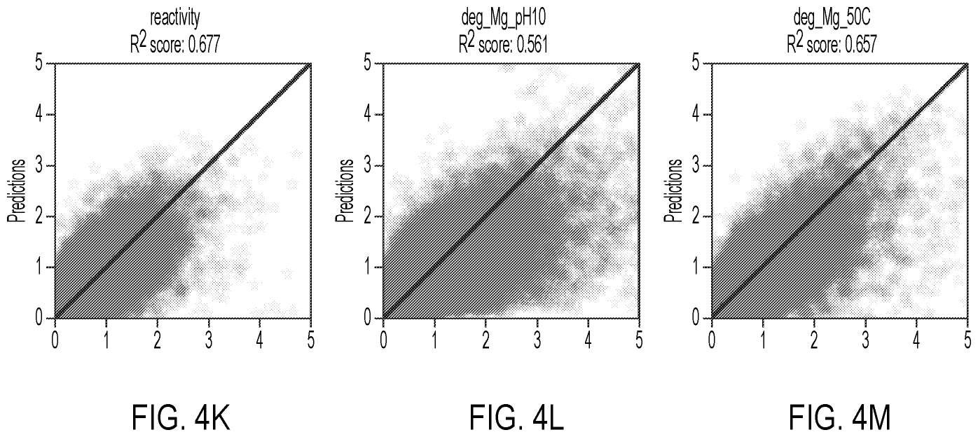

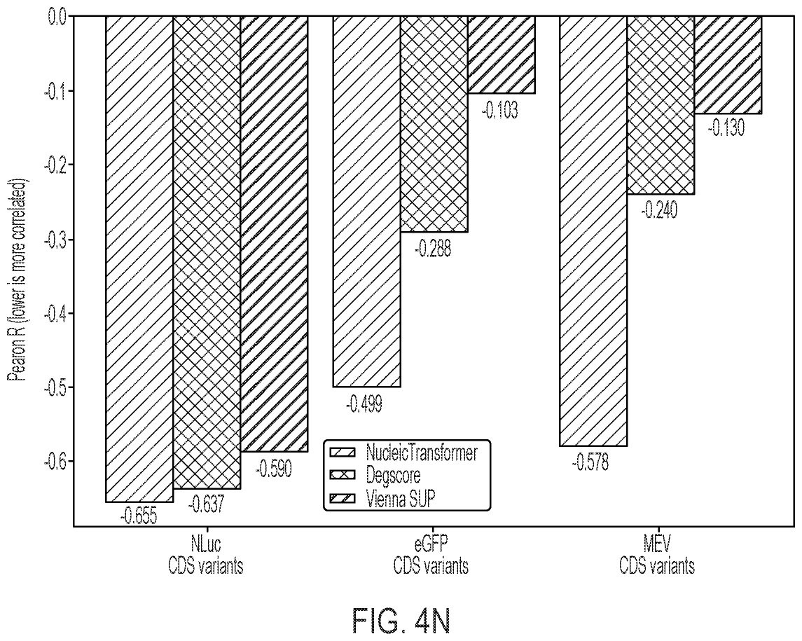

Two sets of models were developed by the inventors to use in the final submissions, one that was trained directly on short sequences with labels and one that was pretrained with all available sequences before training on short sequences with labels. In order to robustly select the desired sequences, the learned attention weights from the transformer encoder were visualized and evaluated (FIGS. 4A-4C). As the BPP matrix and distance matrix were added as biases, both learned attention distributions of pretrained and non-pretrained models resembled the BPP and distance matrix, but there were also some key differences. FIGS. 4A-4C is the visualization of BPP+distance matrix, attention weights of a non-pretrained Nucleic Transformer, and attention weights of a pretrained Nucleic Transformer model, respectively. The non-pretrained model paid heavy attention indiscriminately to pairs of positionally close nucleotides, as indicated by the bright stripes parallel and close to the diagonal of the attention matrix. This indicates the non-pretrained model evaluated positionally close nucleotides as important when making predictions on mRNA degradation properties, which seemed highly unlikely. On the other hand, the pretrained model did not show the same bias towards pairs of positionally close nucleotides, and was able to recognize the weak BPP connections which were barely visible on the original BPP matrix. In this case, the model seemed to make more effective use of the BPP matrix generated by biophysical models. Because of these considerations, the pretrained models were favored for the final submissions, where one submission was an average of 20 pretrained models, and the other was an average of 20 pretrained models and 20 non-pretrained models. Results on the private 176 test set validated our selection based on visual inspection of attention weights (FIG. 4D). Pretrained models performed 177 much better than non-pretrained models on both public test set and private test set. The non-pretrained models would 178 have placed us at 39th/1636 instead of 7th/1636. Notably, the predictions on the private test set have much higher error, 179 likely both due to longer sequence length and more sequence diversity in the private test set.

Semi-Supervised Learning Leads to More Accurate Predictions.

By using predictions generated by an ensemble of 5 Nucleic Transformers models as pseudo-labels, the test set MCRMSE was reduced to 0.33722, compared to the top solution's 0.34198 (FIG. 4D). This is surprising given the that the predictions used as pseudo-labels could only score 0.3438 on the private test set. When the R² scores of supervised only, unsupervised, and semi-supervised learning were compare, the unsupervised and semi-supervised resulted in sizable improvements over the supervised only (FIGS. 4E-4M). Also, predictions for deg_Mg_pH10 have significantly larger errors than the other 2 properties, which is expected because the biophysical models used are incapable of generating different secondary structure predictions at different pHs. It is also important to note that the semi-supervised learning approach requires pseudo-labeling and knowing the test set distribution beforehand. Therefore, this approach would be applicable only in certain circumstances. FIGS. 4E-4G are R² score plots of the predictions versus the ground truth of three properties (reactivity and degradation at pH 10 and at 50° C.) of RNA as best ensemble of semi-supervised learning results on the private test set during the OpenVaccine competition, according to an embodiment. FIGS. 4H-4M are R² score plots of the predictions versus the ground truth of three properties (reactivity and degradation at pH 10 and at 50° C.) of RNA on the private test set during the OpenVaccine competition in three environments: supervised only (FIGS. 4H-4J) and unsupervised (FIGS. 4K-4M). Also, predictions for deg_Mg_pH10 have significantly larger errors for the other 2 properties. This is expected because the biophysical models used are incapable of generating different secondary structure predictions at different pH's. Semi-supervised learning approach requires pseudo-labeling and knowing the test set distribution beforehand.

Nucleic Transformer is a Strong Predictor of mRNA In Vitro Half-Life.

Comparing predictions from the Nucleic Transformer method on groups of CDS variants mRNA design sequences to half-life data produced by Leppek et al., the Nucleic Transformer provides improved correlation (Pearson R=−0.655) with half-lives of the largest group of nanoluciferase CDS variants (928 bp on average) as compared previous models quantifying mRNA degradation such as Degscore (Pearson R=−0.637) and sum unpaired probability (SUP) (Pearson R=−0.58) (FIG. 4N). On smaller groups of CDS variants of eGFP (1191 bp on average) and Multi-Epitope-Vaccine (MEV) (505 bp on average), Nucleic Transformer shows larger improvement (−0.499/−0.578) over Degscore (−0.288/−0.240) and SUP (−0.103/−0.130). Compared to top solutions in OpenVaccine, the Nucleic Transformer generalizes better to longer CDS variants (Nanoluciferase and GFP) (Table 8), both of which are much longer than sequences in the training data.

TABLE 8
Pearson correlation of Nucleic Transformer
predictions with in vitro half-life

| | NLuc Eterna | eGFP | MEV |
|---|---|---|---|
| NucleicTransformer | −0.655 | −0.499 | −0.578 |
| nullrecurrent (1st place) | −0.601 | −0.220 | −0.685 |
| kazuki (2nd place) | −0.623 | −0.376 | −0.597 |
| Degscore | −0.637 | −0.288 | −0.240 |
| Vienna SUP | −0.590 | −0.103 | −0.130 |

Overall, the Nucleic Transformer method is the only method that provides correlation equal to or better than −0.5 across all 3 CDS groups. Computational design of CDS regions of mRNA sequences can lead to close to a threefold increase in in vitro half-life using Degscore, a ridge regression model, following dynamic programming optimization of Gibbs free energy using linear design. While Degscore and linear design leverage either structure information or thermodynamics, the Nucleic Transformer method can simultaneously and effectively utilize the most important features in regards to mRNA degradation: sequence and base pairing probability, as revealed by the leave-one-feature-out ablation studies (LOFO) (FIG. 4O). As a result, the Nucleic Transformer method can help design more stable mRNA sequences.

Provided here are computer-implemented methods for generating and using a neural network model for determining a property of a polynucleotide sequence. The neural network models can be one or more of convolutional neural networks, deep neural networks, recurrent neural networks, deep residual convolutional neural networks, and the like. These neural network-implemented methods and systems process nucleic acid sequences to identify, characterize, and interpret specific properties, like characterization of promoters or enhancers of gene sequences, identification of viral genomes, and half-life of the nucleic acids. The resulting models can be trained in supervised, unsupervised, and semi-supervised environments

One embodiment includes a computer-implemented method for determining a property of a polynucleotide sequence that includes the steps of: receiving a plurality of nucleotides organized by their position defining a polynucleotide sequence and a request for determining a property of the polynucleotide sequence; and applying a deep learning prediction model to the plurality of nucleotides to determine the property of at least a portion of the polynucleotide sequence. The deep learning prediction model applies a trained convolutional neural network and a self-attention mechanism to capture local and global dependencies of each of the plurality of nucleotides and the polynucleotide sequence. The convolutional neural network can be trained in one or more of a supervised, unsupervised, and semi-supervised learning environments. The property of the portion of the polynucleotide sequence can be the presence of a promoter or an enhancer sequence for gene expression. The property of the portion of the polynucleotide sequence can be the presence of a viral gene. The property of the portion of the polynucleotide sequence can be the stability of the polynucleotide sequence under an environmental parameter. The environmental parameter can be one or more of temperature, pH, or presence of a nuclease. Embodiments also include one or more non-transitory, computer-readable medium having computer executable instructions that implement the foregoing computer-implemented methods.

Embodiments of a method also include the steps of receiving a request to improve a property of a polynucleotide sequence to generate a reconfigured polynucleotide sequence with the desired property, querying a database of Nucleic Transformer models to identify a suitable Nucleic Transformer model associated with the desired property; applying the suitable Nucleic Transformer model to predict the current property of the polynucleotide sequence and to present an output of one or more changes to the polynucleotide sequence to improve the current property to the desired property. For example, the polynucleotide sequence can be analyzed by the suitable Nucleic Transformer model to contain a weak promoter sequence and the suitable Nucleic Transformer model can present one or more changes to the polynucleotide sequence to increase the promoter strength. In another example, the polynucleotide sequence can be analyzed by the suitable Nucleic Transformer model to have a half-life of 1 hour at 37° C. and the suitable Nucleic Transformer model can present one or more changes to the polynucleotide sequence to increase the half-life to 6 hours or more at 37° C.

Embodiments also include systems with one or more processors coupled to a memory, which is loaded with computer instructions to perform a computer-implemented method for determining a property of a polynucleotide sequence. These computer instructions, when executed on the one or more processors implement actions that include: receiving a plurality of nucleotides organized by their position defining a polynucleotide sequence and a request for determining a property of the polynucleotide sequence; and applying a deep learning prediction model to the plurality of nucleotides to determine the property of at least a portion of the polynucleotide sequence. The deep learning prediction model applies a trained convolutional neural network and a self-attention mechanism to capture local and global dependencies of each of the plurality of nucleotides and the polynucleotide sequence. The convolutional neural network can be trained in one or more of a supervised, unsupervised, and semi-supervised learning environments. The property of the portion of the polynucleotide sequence can be the presence of a promoter or an enhancer sequence for gene expression. The property of the portion of the polynucleotide sequence can be the presence of a viral gene. The property of the portion of the polynucleotide sequence can be the stability of the polynucleotide sequence under an environmental parameter. The environmental parameter can be one or more of temperature, pH, or presence of a nuclease. Embodiments also include one or more non-transitory, computer-readable medium having computer executable instructions that implement the foregoing computer-implemented methods.

One such method includes the steps of receiving a plurality of nucleotides organized by their position defining a polynucleotide sequence; and applying a deep learning prediction model to the plurality of ribonucleotides to determine a specific property of each nucleotide and the polynucleotide sequence, wherein the deep learning prediction model includes a convolutional neural network and self-attention mechanism to capture local and global dependencies of each nucleotide and the polynucleotide sequence. In an embodiment, properties of single nucleotides are aggregate locally using 1D convolutions before self-attention mechanism was used to obtain a nucleotide to nucleotide attention map. The convolutional neural network can include a 1-D convolution operation or a 2-D convolution operation or a combination of both. The deep learning prediction model can be trained in one or more of a supervised, unsupervised, and semi-supervised learning environments. In an embodiment, the deep learning prediction model receives information from biophysical models. These models include data regarding secondary structures that stabilize a polynucleotide sequence. Provided here are computer-implemented systems and methods for evaluating structure and stability of polynucleotides to develop a RNA sequence directed to expression of one or more immunogenic proteins of a viral envelope.

The methods and systems described herein may be implemented on many different types of processing devices by program code comprising program instructions that are executable by the device processing system. The software program instructions may include source code, object code, machine code, or any other stored data that is operable to cause a processing system to perform the methods and operations described herein. Any suitable computer languages may be used such as C, C++, Java, etc., as will be appreciated by those skilled in the art. Other implementations may also be used, however, such as firmware or even appropriately designed hardware configured to carry out the methods and systems described herein. The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other non-transitory computer-readable media for use by a computer program. The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The disclosure is presented in terms of methods and architectures and symbolic representations of operations on data bits within a device memory. These descriptions and representations are the tools used by those skilled in the data processing arts to most effectively convey the substance of their work to others skilled in the art. An architecture as used here is conceived to be a self-consistent sequence of operations leading to a desired result. Unless specifically stated otherwise as apparent from the disclosure, it is appreciated that throughout the description, discussions utilizing terms such as “receiving,” “determining,” or the like, refer to the action and processes of a device, or similar electronic computing device, that manipulates and transforms data represented as physical (electronic) quantities within the device's registers and memories into other data similarly represented as physical quantities within the device memories or registers or other such information storage, transmission or display devices.

### Datasets

High quality and accessible datasets provide useful and robust information and they establish benchmarks where fair comparisons between different models can be made. Several high quality datasets were used, which include a labeled E. coli promoter/non-promoter dataset, a labeled viral/non-viral genome dataset, and an RNA dataset of COVID-19 vaccine candidates with experimentally measured degradation properties. The E. coli promoter/non-promoter dataset and the labeled viral/non-viral genome dataset have been well studied in the literature, while the RNA dataset is extremely novel and little existing work has dealt with RNA degradation properties.

E. Coli promoter/non-promoter dataset: The E. Coli promoter/non-promoter dataset used is an experimentally confirmed benchmark dataset used in the literature to model and evaluate DNA promoter sequences. All DNA sequences in the dataset were collected from the RegulonDB database (electronic resource about the regulatory network of gene expression in Escherichia coli K-12), and sequences were screened by CD-HIT based on redundant sequence identity. This dataset consists of 2,860 promoter sequences and 2,860 non-promoter sequences. All promoter sequences were experimentally confirmed and collected from the RegulonDB database (version 9.3). The non-promoters sequences were extracted randomly from the middle regions of long coding sequences and convergent intergenic regions in E. coli K-12 genome. This dataset was downloaded from the GitHub service https://github.com/Shujun-He/Nucleic-Transformer/blob/master/src/promoter_classification/v9d3.csv. Model performance on this dataset was evaluated using 5-fold cross validation, where the data was split using iterative stratification. The metrics used for this dataset are accuracy, sensitivity, specificity, and Matthews correlation coefficient (MCC). In addition to cross-validation, an independent test set composed of experimentally verified E. Coli promoters was used that has been recently added to the RegulonDB database. additional non-promoter sequences we extracted (filtered by CD-HIT by 0.8 redundant sequence identity) from the intron and CDS regions of E. coli genome, and datasets with 5:1, 10:1, 20:1 imbalance were constructed.

Human and Mouse Promoter datasets. The TATA/non-TATA human and mouse promoter sequences were downloaded from https://epd.epfl.ch//index.php. Specifically, for each promoter sequence, the −549 to 350 bp region was downloaded so the middle −249 to 50 bp regions were used as positive promoter examples and the two flanking regions as negative examples. Subsequently, positive and negative sets were screened by CD-HIT based on redundant sequence identity using a threshold of 0.8. As the two flanking regions were extracted per promoter sequence, these datasets have roughly twice the number of non-promoters as promoters.

Enhancer/Non-enhancer dataset. The enhancer/non-enhancer dataset was first introduced in the iEnhancer-2L study. Enhancer sequences from nine different cell lines were collected and segmented into 200 bp fragments. With CD-HIT, DNA sequences were filtered to exclude ones with high (>20%) similarity, resulting in 1484 enhancer sequences and 1484 non-enhancer sequences to use for training and validation, and 200 enhancer sequences and 200 non-enhancer sequences to use for testing.

Viral/non-viral genome dataset: The viral/non-viral genome dataset is same as the one used to trained the ViraMiner method, which is a deep learning-based method to identify viruses in various human biospecimens and consists of 19 different NGS experiments analyzed and labeled by PCJ-BLAST following de novo genome assembly algorithms. This dataset is publicly available at https://github.com/NeuroCSUT/ViraMiner. DNA sequences included in this dataset were cut to 300 bp segments with remaining portions smaller than 300 bp discarded. Further, all sequences that contain “N” (unknown with equal probability to any of the four nucleotides) were removed as well. This dataset has approximately 320,000 DNA sequences in total. The main challenge with this dataset is the class imbalance, where only 2% of sequences are of viral origin. The dataset is split into training dataset, validation dataset, and test dataset, where hypertuning was done with the training and validation set, and model performance evaluated on the test set. The metric used for this dataset is AUC (Area Under the Receiver Operating Characteristic Curve).

OpenVaccine Challenge dataset: The mRNA-based compositions are the leading candidate for COVID-19 vaccines, but they face significant limitations, one of which is the spontaneous degradation of mRNA. The OpenVaccine challenge provided participants with 2400 107-bp mRNA sequences with the first 68 base pairs labels with 5 degradation properties at each position. These properties are reactivity, deg_pH10, deg_Mg_pH10, deg_50C, and deg_Mg_50C. More details on these properties can be found at https://www.kaggle.com/c/stanford-covid-vaccine/data. The test set was divided into a public test set and a private test set. The final evaluation was done on a portion of the private test set consisting of 3005 130-bp mRNA sequences, whose degradation measurements were conducted during the 21-day challenge and revealed at the end. The test set was subjected to screening based on three criteria: (1) Minimum value across all 5 degradation properties must be greater than −0.5; (2) Mean signal/noise across all 5 degradation properties must be greater than 1.0. [Signal/noise is defined as mean (measurement value over 68 nts)/mean(statistical error in measurement value over 68 nts)]; and (3) Sequences were clustered into clusters with less than 50% sequence similarity and chosen from clusters with 3 or fewer members. After screening, only 1172 sequences remained on the test set. Final evaluation was done on 3 of the 5 properties (reactivity, deg_Mg_pH10, and deg_Mg_50C). Unlike the training set, the test set has longer mRNA sequences with more sequence diversity and

$$MCRMSE = \frac{1}{{N}_{i}} {\sum }_{j = 1}^{{N}_{i}} \sqrt{{\frac{1}{n} {\sum }_{i = 1}^{n} ( {y}_{ij} ) - {\hat{y}}_{ij} )}^{2}} .$$

more measurements (first 91 positions) per sequence; in fact, more predictions had to be made for the test set than there were training samples. The metric used for ranking in the competition is MCRMSE (mean columnwise root mean squared error):

Where Nₜ is the number of columns, n the number of positions predicted, y the ground truth, and y\hat{ } the predicted value. In addition, R² score (coefficient of determination) was also used during further analysis.

DeepSea dataset. The DeepSea dataset was first compiled by Zhou and Troyanskaya. Training labels were computed from uniformly processed ENCODE and Roadmap Epigenomics data releases. The genomes were split into 200-bp bins and bins with at least one TF binding event were kept, totaling 512,636,200 bp of sequences. Each training sample is a 1000-bp sequence from the human GRCCh37 reference genome centered on each 200-bp and is labeled with 919 chromatin features. Training and testing sets were split by chromosomes in a strictly non-overlapping fashion. Chromosome 8 and 9 were kept out of the training set to test chromatin feature prediction performances. Area under the receiver operating 259 characteristic curve is used to measure performance.

### Architecture Design

K-mers with 1-D convolutions: A common method to process DNA sequences in the field of bioinformatics is to transform them into k-mers. For instance, consider a short DNA sequence ATGC. The k-mers in this sequence are subsequences of length k, so ATGC has three 2-mers AT, TG, and GC, two 3-mers ATG and TGC, and one 4-mer ATGC. Converting a DNA sequence into k-mers is analogous to separating a language sentence into words and allows for more efficient extraction of information from DNA. The extraction of k-mers from a DNA sequence can be considered a sliding window of size k taking snapshots of the sequence while moving one position at a time from one end of the sequence to the other, which is conceptually identical to the convolution operation used in deep learning. Consider a simple example of convolution involving a vector S∈R^{l}, where l is the length of the vector, and a convolution kernel K∈R³, which convolves over the vector S. If the convolutional kernel strides one position at a time, an output vector of dot products O∈R^{l-2} is computed,

$${O}_{p} = {\sum }_{i ∈ \{ 0 , 1 , 2 \}} {K}_{i} {S}_{p + i} ,$$

where p denotes the position in the output vector. In this embodiment, the convolution operation aggregates local information with 3 positions at a time, so if S is a sequence of nucleotides, the convolution operation extracted 3-mers from the DNA sequence S. Consequently, a convolution kernel of size k can be used to transform a DNA sequence into k-mers.

As an embodiment of the model takes sequences of DNA/RNA nucleotides as input, each nucleotide was transformed into embeddings of fixed size d_{model}. For each sequence, a tensor I∈R^{l×dmodel} was determined, where l is the length of the sequence. Because the transformer encoder architecture is being utilized, which is permutation invariant, positional encoding was added:

PE_{(pos,2i)}=sin(pos/5000^{2i/d}^{_{model}})

PE_{(pos,2i+1)}=sin(cos/5000^{2i/d}^{_{model}})

where pos is the position and i is the channel dimension. To create k-mers, convolutions on the tensor I were performed without padding and stride=1. When the convolution operation with kernel size k was performed over I, a new tensor Kₖ∈R^{(I-k+1)×dmodel} representing the sequence of k-mers was generated. A collection of k-mers were obtained, where each k-mer is represented by a feature vector of size d_{model}. The 1D convolution layers were followed by a layer normalization layer.

Indeed, the representation of k-mers deviated from conventional representation of words in deep learning, where each word in the vocabulary directly corresponds to a feature vector in a look up table. The disadvantage of using look up tables for k-mers is that a very small percentage of all possible k-mers are present in any dataset, and there is no way for the network to generalize to unseen k-mers. For instance, if a common promoter motif TATAAT appears in the dataset but a similar motif TATATT does not, there is no way for the network to generalize to the unseen motif TATATT, but by using convolutions, the network is able to recognize that there is not much difference between TATAAT and TATATT, leading to better generalization. Additionally, embeddings of k-mers of larger sizes require a prohibitively large amount of parameters, as the total possible amount of k-mers for a given k is 4^{k}.

Transformer encoder: For the encoder, the vanilla transformer encoder was implemented, which used the multi-head self-attention mechanism. First, the k-mer representations (each k-mer is represented by a feature vector of size d_{model}) are linearly projected into lower dimensional (d_{model}/n_{nhead}) keys, values and queries for n_{nhead} times. Next, the self-attention function is computed with the lower dimensional keys, values and queries for n_{nhead} times independently. In this case, the self-attention function essentially computes a pairwise interaction matrix relating every k-mer to every k-mer (including self to self-interaction) and computes a weighted sum of the values. Unlike a single-head attention function, the multi-head self-attention function linearly projected d_{model}-dimensional keys, values and queries into lower dimensional representations. Then, the multi-head attention function directly operated on the entire sequence. The multi-head mechanism allowed different heads to learn different hidden representations of the input, leading to better performance. The multi-head self-attention mechanism can be summarized in a few equations:

$$Attention ( Q , K , V ) = softmax ( \frac{{QK}^{T}}{\sqrt{{d}_{k}}} ) V$$

$$MultiHead ( Q , K , V ) = Concat ( {head}_{1} , … , {head}_{h} ) {W}^{O}$$

$$where {head}_{i} = Attention ( {QW}_{i}^{Q} , {KW}_{i}^{K} , {VW}_{i}^{V} ) .$$

As only the transformer encoder was used, Q, K, V came from the same sequence of feature vectors (hence the name self-attention), each of which represents a k-mer with positional encoding. The self-attention mechanism enables each k-mer to attend to all k-mers (including itself), so global dependencies are drawn between k-mers of any size at any distance. Contrary to recurrence and convolutions, both of which enforce sparse local connectivity, transformers allowed for dense or complete global connectivity. The ability to draw global dependencies of transformers is a huge advantage over recurrence and convolutions, both of which struggle with long sequences. The self-attention function is followed by a position-wise feedforward network applied separately and identically to each position:

FFN(x)=ReLU(xW₁+b1)W₂+b₂.

The position-wise feedforward network consists of two linear transforms with a ReLU activation in between. Conventionally, the combination of self-attention and position-wise feedforward network is referred to as the transformer encoder layer, and a stack of transformer encoder layers is referred to the transformer encoder.

Incorporating Biophysical Models to Predict RNA Degradation.

Although it is sufficient to use sequence information for DNA tasks, predicting degradation of RNA requires more than just sequence information. Firstly, the predicted structure per position were included, which described whether a nucleotide is paired or unpaired with another one via hydrogen bonding. The predicted structure is generated using arnie with log_gamma set to 0 (arnie is available at https://github.com/DasLab/arnie). Also, the predicted loop type assigned by bpRNA was included. Two embedding layers were added to represent structure and loop type, and the resulting feature vectors were concatenated and the dimensionality reduced with a linear transformation. Additionally, a modified version of the base-pairing probability matrix was directly added into the attention function (note that this is a modified version of the prior Attention equation with M_{bpp} as an additional input):

$$Attention ( Q , K , V , {M}_{bpp} ) = softmax ( \frac{{QK}^{T}}{\sqrt{{d}_{k}}} + γ {M}_{bpp} ) V$$

where γ is a learnable parameter and M_{bpp} is the modified base-pairing probability matrix. The original base-pairing probability matrix contains the probabilities for every possible base pair in an RNA sequence and has been used for many RNA informatics tasks. Here in addition to base-pairing probabilities, inverse, inverse squared, and inverse cubed pairwise distance matrices were also stacked on top of the original base-pairing probability matrix, where the distance is the number of covalent bonds between the pair of nucleotides (this can also be considered the path length in an RNA graph where the only edges are the covalent bonds). The inverse distance matrices encoded certain information about the relative distance between pairs of nucleotides, as pairs of nucleotides with a small number of covalent bonds in between are likely to be closer to each other spatially. Because the distance matrix already encodes information about position, positional encoding for mRNA was not used.

Because 1-D convolution operation used in the Nucleic Transformer method does not use padding, the convolution product ends up with reduced dimensionality in the L dimension when the convolution kernel size is bigger than 1. As a result, the base pairing probability matrix cannot be directly added to self-attention matrix. To circumvent this, 2D convolution was performed with the same kernel size as the 1D convolution on the modified base pairing probability matrix without padding, so the dimensionality of the feature map became C×(L−k+1)×(L−k+1). The attention function now is:

$$Attention ( Q , K , V , {M}_{bpp} ) = softmax ( \frac{{QK}^{T}}{\sqrt{{d}_{k}}} + γ conv 2 d ( {M}_{bpp} ) ) V .$$

Conceptually, instead of a base pair to base pair interaction mapping, the 2D convolution product of the modified base pairing probability matrix can be seen as a k-mer to k-mer pairwise interaction mapping with matching dimensionality to the 1D convolution k-mer products. Aside from matching dimensionality, the 2D convolution operation also made up for some missing information regarding the geometry of mRNA folding. To illustrate this, an mRNA sequence in the OpenVaccine dataset (FIG. 2) was visualized to explain the physical and mathematical reasoning behind the 2D convolution operation. FIG. 2 is an illustration of the physical meaning of the two-dimensional (2D) convolution on BPP matrix that presents a visualization of the folding of sequence id_0049f53ba, according to an embodiment. While inspecting the interaction between A-20 (A at position 20), G-21, C-40, and U-41, it was observed that A-20 and U-40 are quite close to each other and there is some degree of interaction between them, despite A-20 and U-40 not forming hydrogen bonds. However, looking at the portion of BPP matrix and distance matrix corresponding to the 2×2 connection between A-20 (A at position 20), G-21, C-40, and U-41, it was observed that neither the BPP matrix nor the distance matrix conveyed this information, as the component (40,20) has zero or close to zero values on both the BPP matrix and the distance matrix. When a 2×2 convolution kernel operated on the BPP matrix and distance matrix (for illustration purposes here we simply draw a kernel with all values set to unity), it essentially fused the 4 connections between A-20, G-21, C-40, and U-41, and created a strong connection between the 2 2mers (A-20, G-21 and C-40, U-41). Therefore, it is easier for the network to learn the interaction between A-20 and G-40 (as well as for G-21 and U-41).

The combination of convolution and self-attention cannot produce nucleotide position wise predictions, as it generated k-mer encodings instead single base pair encoding. In order to make predictions per nucleotide position, additional deconvolution layers were introduced to retrieve full dimensional encodings, which allowed residual connections of both 1D and 2D encodings before and after the transformer encoder. As result, both the single nucleotide embeddings and the modified BPP matrix underwent deep transforms before providing predictions.

The modified Nucleic Transformer architecture used for the RNA task is summarized in FIG. 3B, which can be seen as a special case of a series of multiple Nucleic Transformers with a single transformer encoder layer followed by a deconvolution layer. FIG. 3B is diagrammatic representation of the Nucleic Transformer stack that receives input information from biophysical models. Also, because the OpenVaccine challenge required making predictions at each position of the RNA sequence, it was important for the last transformer encoder layer right before providing predictions to operate on single nucleotide encodings instead of k-mer encodings. A method was developed to construct the stack of the Nucleic Transformer methods with two main hyperparameters k and m_{layer}, set equal to each other (FIG. 3B). The first single layer Nucleic Transformer has k=m_{layer}, and the size of the convolution kernel was decreased by 1 for the next single layer Nucleic Transformer. Therefore, when the Nucleic Transformer method in the stack is reached, k becomes 1 and the last Nucleic Transformer is simply a transformer encoder layer with an added bias from the BPP feature map.

Training Details

Optimizer and training schedule. For DNA classification tasks, the Adam adaptive learning rate optimization algorithm was used with β1=0.9, β2=0.99, and ∈=1e−8. Weight decay is set to 1e−5. The learning rate schedule is a stepwise inverse square root decay schedule with warm up. As relatively small batch sizes were used during training, the learning rate was adjusted by a scaling factor C:

learning rate=C·d_{model}^{-0.5}·min(step_num^{-0.5},step_num·warmup_steps^{-1.5}).

In an embodiment, C is set 0.1 and warmup_steps is set to 3200. Additionally, dropout of probability 0.1 was used in all attention layers, fully connected layers, and positional encoding.

For the RNA task, the Adam algorithm was found to be underfitting. Therefore, the Ranger algorithm was used, which uses gradient centralization from https://github.com/lessw2020/Ranger-Deep-Learning-Optimizer. As for the training schedule, a flat and anneal method was used, where training starts with a flat learning rate of 1e−3 and then 75% through all the epochs training proceeds with cosine annealing schedule reducing learning rate down to 0 at the end of training. Weight decay is set to 0.1.

Error weighted loss: Because the OpenVaccine dataset came from experimental measurements that had errors, the losses were adjusted based on the error for each measurement during supervised training:

error adjusted loss=loss×(α+e^{β×error})

where α and β are tunable hyperparameters. If α is set to 1 and β to infinity, then the loss values stay the same; otherwise gradients from measurements with large errors would be lowered to prevent the neural network from overfitting to experimental errors. For the OpenVaccine dataset, α=0.5 and β=5 were used.

Usage of biophysical models. Secondary structures predicted by an ensemble of biophysical models were used including RNA soft, rnastructure, CONTRAfold, Eternafold, nupack, and Vienna. For each sequence, secondary structure predictions were generated at 37 and 50 Celsius, as two of the scored degradation properties were measured at different temperatures. Although predictions for a degradation property at pH10 were needed, none of the biophysical models used could generate predictions at different pH's. With 6 packages, 12 secondary structure predictions for each sequence were generated. During training, one of the 12 secondary structure predictions was randomly selected for each sample during a forward and backward propagation pass. During validation and testing, the averaged predictions used all 12 secondary structure predictions. Performance of the Nucleic Transformer models using different packages and ensembled together (Table 9).

TABLE 9
Performance using inputs generated by different biophysical models
to predict mRNA degradation in the Open Vaccine dataset.

| | | | Public | Private |
|---|---|---|---|---|
| | | | MCRMSE | MCRMSE |
| | Public | Private | semi- | semi- |
| Package | MCRMSE | MCRMSE | supervised | supervised |
| RNAsoft | 0.23154 | 0.34521 | 0.23101 | 0.33841 |
| rnastructure | 0.23637 | 0.34639 | 0.23502 | 0.33989 |
| EternaFold | 0.23158 | 0.34613 | 0.23081 | 0.33878 |
| Contrafold_2 | 0.23245 | 0.34858 | 0.23123 | 0.34111 |
| NUPACK | 0.23679 | 0.34955 | 0.23587 | 0.34292 |
| Vienna | 0.23492 | 0.34515 | 0.2337 | 0.33864 |
| avg of all | 0.22976 | 0.34375 | 0.22914 | 0.33722 |

In certain embodiments, predictions made using secondary structure information from rnastructure have the lowest error on the private test set without semi-supervised learning, although RNAsoft, rnastructure, eternafold, and nupack all give comparable results. With semi-supervised learning, Vienna gives most accurate predictions, while RNAsoft, rnastructure, eternafold come close. By averaging all predictions made using all available packages on the private test set, the error was always lower than using any single package, indicating that ensembling different biophysical models is a good strategy that would give better predictions than any single model.

Random mutations during training. It is known that deep learning models can perfectly memorize completely random data and labels, so to combat the memorization effect, noise was injected artificially by randomly mutating positions in the source DNA/RNA sequence before forward and backward propagation during training. This injection of random noise is done during DNA supervised learning and RNA unsupervised learning. In the embodiments here, nucleotides were randomly mutated in randomly selected positions, and because nucleotide at each selected position was not ensured to be changed, the average amount of mutations is ¾ of nₘᵤₜₑ. It is true that these random mutations could be non-label-preserving; however, deep learning algorithm is robust to massive label noise, so the non-label-preserving mutations should simply be ignored by the network during training. The number of positions to randomly mutate is a hyperparameter that is noted as nₘᵤₜₑ, which was optimized to determine the best value.

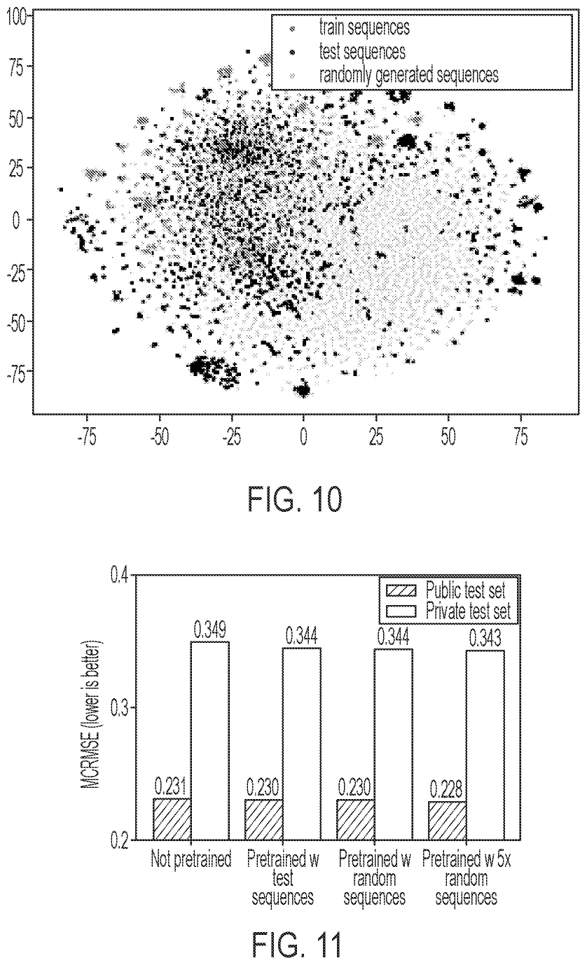

Learning from random sequences. To explore whether the pretraining performance gain results from knowing the test set distribution, additional completely random sequences were also generated to use for pretraining. The sequences in the test set have more sequence diversity than the training set, and generated sequences were even more diverse (FIG. 10). FIG. 10 is a t-distributed stochastic neighbor embedding (t-SNE) plot of RNA sequences in the training set, test set, and randomly generated set. The pretraining process was repeated with random sequences using the same hyperparameters. Pretraining with random sequences instead of test sequences results in almost identical performance and increasing the amount of random sequences for pretraining leads to slightly better performance on both test sets (FIG. 11). FIG. 11 is a graphical representation of a test set performance of OpenVaccine challenge using different pretraining procedures. These results suggest that pretraining improves test set error not because of information leak from knowing the test sequences but rather training the model regarding the generalized rules of mRNA secondary structure formation.

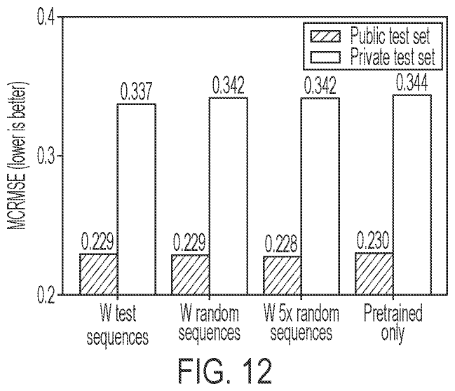

Additionally, random sequences were also pseudo-labeled and the models were retrained in semi-supervised fashion. In this case, pseudo-labeling with completely random sequences did not lead to significant improvements (FIG. 12). FIG. 12 is a graphical representation of a Semi-supervised learning with test sequences compared with random sequences. It is likely that due to the large mutation distance of random sequences compared to train and test sequences, the pseudo-labels on the random sequences are not close to ground truth values at all. Therefore, the semi-supervised learning process ended up feeding mostly noise to the model during training, although it did lead to very slight improvement on the private test set over the model without semi-supervised learning.

Best Hyperparameters for Different Tasks.

For E. coli promoter classification, the best results were obtained using k=7, nₘᵤₜₑ=45, six transformer encoder layers, d_{model}=256, n_{head}=8, and batch size is 24. For human and mouse promoters, the best results were obtained using k=11, nₘᵤₜₑ=45, six transformer encoder layers, d_{model}=256, n_{head}=8, and batch size 64. For the DeepSea dataset, 2 more cony 1 d layers and 3 maxpooling layers were introduced to reduce the input sequence length to transformer encoder, best results were obtained used an ensemble of k=7 and k=9, d_{model}=1024, n_{head}=16. For enhancer predictions, the same hyperparameters as for E. coli promoters were used. For viral/non-viral DNA classification, the best results were obtained using nₘᵤₜₑ=40, six transformer encoder layers, d_{model}=512, and n_{head}=8. For models trained during the OpenVaccine competition, n_{head} is set to 32, &odd is set to 256, dropout is set to 0.1, and conv2d filter size is set to 32, α and β are both 1. Half of the models were trained with sequences with signal to noise greater than 1 and half were trained with signal to noise greater than 0.5. Results are better when penalizing measurements with high error more by reducing a to 0.5 and increasing β to 5. Although using more models at more different conditions can give better results, for better reproducibility we only trained 5 models with k=n_{layer}=3, 4, 5, 6, 7, 8 for each experiment hereinafter. Here n_{head} is set to 32, d_{model} is 256, dropout is set to 0.1, and conv2d filter size is set to 8. Sequences with signal to noise greater than 0.25 were only used for training and signal to noise greater than 1 for 10-fold cross validation.

### Data Availability

The E. coli promoter dataset can be downloaded at https://github.com/Shujun-He/Nucleic-Transformer/blob/master/src/promoter_classification/v9d3.csv. The E. coli promoter datasets with additional non-promoter sequences can be downloaded from https://oup.silverchair-cdn.com/oup/backfile/Content_public/Journal/bioinformatics/36/19/10.1093_bioinformatics_bta a609/1/btaa609_supplementary_data.zip?Expires=1645689990& Signature=zFj-ZRk0dzxou1wNqh2KIQ0iCOVc7v8ipBGwRFtyM4aAv4T7FwC2oh4oedS˜G444Rj74iy9s1m3j M7TONBgrCxi2O1v1IomJKx6e3AhHK44Xr2X˜Vr4NpE2V6RozztTEuSiyBPTUFhAXyd6mos COk7LXQifNeUA˜jTBNaGhrh0KjHTacZDB5eAPTesW6Bo4tb4LyEjyXGH3tU-2hnwVe9ujoAeDi4fvnE1FwO5eVWVUrPbNdNcKveOD1SsP5W1ktSB52B WJFtaWrR76n0jSK x4YmA˜rN8Mu˜X-EWyI1a-xF1RIorUU8eaxps2nVtc5Tfr3NE9dWAkQWOJLVSJvGm1Q &Key-Pair-Id=APKAIE5G5CRDK6RD3PGA. The Deep Sea dataset was downloaded from http://deepsea.princeton.edu/media/code/deepsea_train_bundle.v0.9.tar.gz. The enhancer 468 dataset is downloaded from https://github.com/khanhlee/bert-enhancer/tree/main/data. The dataset of viral DNA can be downloaded from https://github.com/NeuroCSUT/ViraMiner. OpenVaccine dataset is available at https://www.kaggle.com/c/stanford-covid-vaccine/data. Pretrained models can be accessed from Kaggle notebooks: https://www.kaggle.com/shujun717/nucleic-transformer-promoter-inference, https://www.kaggle.com/shujun717/nucleic-transformer-virus-inference, https://www.kaggle.com/shujun717/nucleic-transformer-rna-degradation-inference.

### Copyrighted Code Availability

All training code to fully reproduce results is released at https://github.com/Shujun-He/Nucleic-Transformer, and a web application was developed using H2O.ai's wave and is available at https://github.com/Shujun-He/Nucleic-Transformer-WebApp

The processes and displays presented herein are not inherently related to any particular device or other apparatus. Various general-purpose systems may be used with programs in accordance with the teachings herein, or it may prove convenient to construct a more specialized apparatus to perform the operations described. The required structure for a variety of these systems will be evident from the disclosure and the drawings. In addition, the disclosure is not described with reference to any particular programming language. It will be appreciated that a variety of programming languages may be used to implement the teachings of the disclosure as described herein.

In this disclosure, specific exemplary embodiments have been described. It will be evident that various modifications may be made to those embodiments without departing from the broader spirit and scope set forth in the following claims. The specification and drawings are, accordingly, to be regarded in an illustrative sense rather than a restrictive sense.

What is claimed is:

1. A computer-implemented method for determining a property of a polynucleotide sequence, the method comprising:
receiving a plurality of nucleotides organized by their position defining a polynucleotide sequence and a request for determining a property of the polynucleotide sequence; and
applying a deep learning prediction model to the plurality of nucleotides to determine the property of at least a portion of the polynucleotide sequence, wherein the deep learning prediction model applies a trained convolutional neural network and a self-attention mechanism to capture local and global dependencies of each of the plurality of nucleotides and the polynucleotide sequence.

2. The method of claim 1, wherein the property of the portion of the polynucleotide sequence is presence of a promoter sequence for gene expression.

3. The method of claim 1, wherein the property of the portion of the polynucleotide sequence is presence of an enhancer sequence for gene expression.

4. The method of claim 1, wherein the property of the portion of the polynucleotide sequence is presence of a viral gene.

5. The method of claim 1, wherein the property of the portion of the polynucleotide sequence is stability of the polynucleotide sequence under an environmental parameter.

6. The method of claim 5, wherein the environmental parameter is one or more of temperature, pH, or presence of a nuclease.

7. The method of claim 1, the convolutional neural network is trained in one or more of a supervised, unsupervised, and semi-supervised learning environments.

8. A non-transitory, computer-readable medium having computer executable instructions that implement the computer-implemented method of claim 1.

9. A system including one or more processors coupled to a memory, the memory loaded with computer instructions to perform a computer-implemented method for determining a property of a polynucleotide sequence, the computer instructions, when executed on the one or more processors implement actions, comprising:
receiving a plurality of nucleotides organized by their position defining a polynucleotide sequence and a request for determining a property of the polynucleotide sequence; and
applying a deep learning prediction model to the plurality of nucleotides to determine the property of at least a portion of the polynucleotide sequence, wherein the deep learning prediction model applies a trained convolutional neural network and a self-attention mechanism to capture local and global dependencies of each of the plurality of nucleotides and the polynucleotide sequence.

10. The system of claim 9, wherein the property of the portion of the polynucleotide sequence is presence of a promoter sequence for gene expression.

11. The system of claim 9, wherein the property of the portion of the polynucleotide sequence is presence of an enhancer sequence for gene expression.

12. The system of claim 9, the property of the portion of the polynucleotide sequence is presence of a viral gene.

13. The system of claim 9, wherein the property of the portion of the polynucleotide sequence is stability of the polynucleotide sequence under an environmental parameter.

14. The system of claim 13, wherein the environmental parameter is one or more of temperature, pH, or presence of a nuclease.

15. The system of claim 9, the convolutional neural network is trained in one or more of a supervised, unsupervised, and semi-supervised learning environments.
